(19) United States
(12) Patent Application Publication
(10) Pub. No.: US 2017/0345173 A1
(43) Pub. Date: Nov. 30, 2017

(54) Methods for Analyzing Absorbent Articles

(71) Applicant: The Procter & Gamble Company, Cincinnati, OH (US)

(72) Inventors: Jennifer Joan Gustin, Cincinnati, OH (US); David John Maltbie, Hamilton, OH (US)

(21) Appl. No.: 15/601,027

(22) Filed: May 22, 2017

Related U.S. Application Data
(60) Provisional application No. 62/340,572, filed on May 24, 2016.

(51) Int. Cl. G06T 7/521; G06T 7/174; G06T 7/00; G06K 9/00; A61F 13/15

(52) U.S. Cl. CPC G06T 7/521; G06T 7/0008; G06K 9/00201; G06T 7/97; G06T 7/174; A61F 13/15203

(57) ABSTRACT

Methods of analyzing fit and appearance of an absorbent article. The method may include marking a panelist with markers and positioning the panelist in an orientation. The panelist may then be imaged with an image capture device. From the data gathered by the image capture device a first three dimensional surface representation may be generated. The panelist may also be imaged with an absorbent article. The panelist wearing the absorbent article may be placed in the orientation and imaged with the image capture device. The data gathered during the second imaging session may be used to generate a second three dimensional surface representation. Each of the first and second three dimensional surface representations may be aligned using the markers. The first and second three dimensional surface representations may be compared to one another to obtain information on volume, surface area, length, perimeter, and other characteristics.

### FIELD

The present specification generally relates to methods for analyzing absorbent articles and, more specifically, methods for analyzing absorbent articles using at least two three dimensional surface representations.

### BACKGROUND

Observing and/or analyzing the interior of various consumer products can provide information to the product's manufacturer or others. For example, the manufacturer may want to observe the interior of the product and its constituents or components at any of several stages, including during product design, during manufacture, after the manufacture but before the product is packaged, when the product is in its packaging, upon opening of the package, upon removal of the product from the packaging, when the product is in actual or simulated use conditions, etc. Given that products and components thereof may exhibit different characteristics in a wet state versus a dry state, it may be desirable to have a method available which can allow observation and/or analysis of the same product in a dry state, then after the introduction of liquid, in a wet state, and after the introduction of additional liquid, a yet wetter state, and so on. However, observing and/or analyzing the interior of consumer products has traditionally been done with computed tomography (CT), magnetic resonance imaging (MRI), and/or ultrasound. Each of these methods may not practically be done on individuals, but rather, is more practical for mannequins. For example, CT scanning exposes individuals to ionizing radiation. MRI scanning involves restrictive, noisy environments and is relatively costly. Further, ultrasound scanning requires contact with the article and imaging gel which would likely alter the article.

Accordingly, a need exists for alternative methods for observing and analyzing absorbent articles.

### SUMMARY

The present disclosure relates to methods and apparatuses for analyzing absorbent articles, and more particularly, methods and apparatuses for analyzing absorbent article using two or more three dimensional surface representations of a panelist and/or an absorbent article. In some embodiments, a method of analyzing fit and appearance of an absorbent article may include: marking a panelist using a marking protocol, wherein the marking protocol comprises placement of three or more markers on an external surface of the panelist; positioning the panelist in an orientation; imaging the external surface of the panelist with an image capture device, wherein the image capture device uses visible light or an infrared light; generating a first three dimensional surface representation of the surface of the panelist using a computer system; preparing an absorbent article; imaging the external surface of the panelist wearing the absorbent article with the image capture device; generating a second three dimensional surface representation of the surface of the panelist wearing the absorbent article using the computer system; identifying at least three of the three or more markers present in the first three dimensional surface representation of the panelist; selecting the at least three of the three or more markers present in the first three dimensional surface representation using the computer system; identifying at least three of the three or more markers present in the second three dimensional surface representation of the panelist wearing the absorbent article; selecting the at least three of the three or more markers present in the second three dimensional surface representation using the computer system; aligning, using the computer system, at least three of the three or more markers of the first three dimensional surface representation with at least three of the three or more markers of the second three dimensional surface representation to form an overlapping three dimensional surface representation; and comparing the first three dimensional surface representation and the second three dimensional surface representation.

In some embodiments, a method of analyzing fit of an absorbent article disposed on a panelist may include: preparing a first absorbent article; marking a panelist wearing the first absorbent article using a marking protocol, wherein the marking protocol includes placement of three or more markers on an external surface of at least one of the panelist and the first absorbent article; positioning the panelist in an orientation; imaging, using an image capture device and a computer system, the external surface of the panelist wearing the first absorbent article to generate one or more first images, wherein the image capture device uses a visible light and/or an infrared light; using a computer modeling system to generate a first three dimensional surface representation of the surface of the panelist from the one or more first images; preparing a second absorbent article; imaging, using the image capture device and the computer system, the external surface of the panelist wearing the second absorbent article to generate one or more second images; using the computer modeling system to generate a second three dimensional surface representation of the surface of the panelist wearing the second absorbent article from the one or more second images; identifying the three or more markers present in the first three dimensional surface representation of the panelist wearing the first absorbent article; identifying the three or more markers present in the second three dimensional surface representation of the panelist wearing the second absorbent article; aligning, using the computer modeling system, the three or more markers of the first three dimensional surface representation with the three or more markers of the second three dimensional surface representation to form an overlapping three dimensional surface representation; and comparing the first three dimensional surface representation and the second three dimensional surface representation.

In yet some embodiments, a method of analyzing fit of an absorbent article disposed on a panelist may include: marking a panelist wearing a first absorbent article using a marking protocol, wherein the marking protocol includes placement of three or more markers on an external surface of at least one of the panelist and the first absorbent article; positioning the panelist in an orientation; imaging the external surface of the panelist wearing the first absorbent article using an image capture device to generate one or more first images, wherein the image capture device comprises one or more digital cameras, and wherein the image capture device uses visible light and/or infrared light; generating a first three dimensional surface representation of the surface of the panelist using a computer system and the one or more first images; preparing the first absorbent article; imaging the external surface of the panelist wearing the first absorbent article using the image capture device to generate one or more second images; generating a second three dimensional surface representation of the surface of the panelist wearing the first absorbent article using the computer system and the one or more second images; identifying the three or more markers present in the first three dimensional surface representation of the panelist; identifying the three or more markers present in the second three dimensional surface representation of the panelist wearing the first absorbent article; aligning, using the computer system, the three or more markers of the first three dimensional surface representation with the three or more markers of the second three dimensional surface representation to form an overlapping three dimensional surface representation; and comparing the first three dimensional surface representation and the second three dimensional surface representation.

These and additional features provided by the embodiments described herein will be more fully understood in view of the following detailed description, in conjunction with the drawings.

### BRIEF DESCRIPTION OF THE DRAWINGS

The embodiments set forth in the drawings are illustrative and exemplary in nature and not intended to limit the subject matter defined by the claims. The following detailed description of the illustrative embodiments can be understood when read in conjunction with the following drawings, where like structure is indicated with like reference numerals and in which:

FIG. 1 is a perspective view of an absorbent article;

FIG. 2A is a partially cut away plan view of the absorbent article shown in FIG. 1;

FIG. 2B is a cross-sectional view of the absorbent article of FIG. 2A taken along line 2B-2B of FIG. 2A;

FIG. 2C is a cross-sectional view of the absorbent article of FIG. 2A taken along line 2C-2C of FIG. 2A;

FIG. 3 is a partially cut away plan view of an absorbent article;

FIG. 4 is a partially cut away plan view of an absorbent article;

FIG. 5 is a perspective view of an absorbent article;

FIG. 6 is a partially cut away plan view of an absorbent article;

FIG. 7 schematically depicts an example flow chart of a process for analyzing fit and appearance of an absorbent article;

FIG. 8 is a perspective view of a panelist in accordance with one non-limiting embodiment of the present disclosure;

FIG. 9A is a schematic top view of image capture devices placed about a panelist in accordance with one non-limiting embodiment of the present disclosure;

FIG. 9B is a schematic top view of image capture devices placed about a panelist in accordance with one non-limiting embodiment of the present disclosure;

FIG. 10A is a schematic perspective view of a three dimensional surface representation in accordance with one non-limiting embodiment of the present disclosure;

FIG. 10B is a schematic perspective view of a three dimensional surface representation in accordance with one non-limiting embodiment of the present disclosure;

FIG. 11 is a schematic perspective view of a three dimensional surface representation including a plane in accordance with one non-limiting embodiment of the present disclosure;

FIG. 12A is a schematic perspective view of a trimmed three dimensional surface representation of a panelist in accordance with one non-limiting embodiment of the present disclosure;

FIG. 12B is a schematic, bottom, perspective view of a trimmed three dimensional surface representation of a panelist in accordance with one non-limiting embodiment of the present disclosure;

FIG. 13 is a schematic perspective view of two trimmed three dimensional surface representations and an overlapping three dimensional surface representation in accordance with one non-limiting embodiment of the present disclosure;

FIG. 14A is a schematic perspective view of an overlapping three dimensional surface representation in accordance with one non-limiting embodiment of the present disclosure;

FIG. 14B is a schematic perspective view of a portion of the overlapping three dimensional surface representation of FIG. 14A in accordance with one non-limiting embodiment of the present disclosure;

FIG. 14C is a schematic perspective view of a three dimensional surface representation including a cutting plane in accordance with one non-limiting embodiment of the present disclosure;

FIG. 14D is a schematic perspective view of a three dimensional surface representation including a cutting plane in accordance with one non-limiting embodiment of the present disclosure;

FIG. 14E is a schematic perspective view of a three dimensional surface representation including a cutting plane in accordance with one non-limiting embodiment of the present disclosure;

FIG. 15A is a schematic perspective view of a three dimensional surface representation including a slice in accordance with one non-limiting embodiment of the present disclosure;

FIG. 15B is a schematic perspective view of a three dimensional surface representation including a slice in accordance with one non-limiting embodiment of the present disclosure;

FIG. 16 is a schematic perspective view of an overlapping three dimensional surface representation that has been trimmed in the vertical direction in accordance with one non-limiting embodiment of the present disclosure;

FIG. 17A is a schematic perspective view of a three dimensional surface representation in accordance with one non-limiting embodiment of the present disclosure;

FIG. 17B is a schematic perspective view of a three dimensional surface representation in accordance with one non-limiting embodiment of the present disclosure;

FIG. 18A is a schematic perspective view of a three dimensional surface representation including a feature in accordance with one non-limiting embodiment of the present disclosure; and

FIG. 18B is a schematic perspective view of a three dimensional surface representation including a feature in accordance with one non-limiting embodiment of the present disclosure.

### DETAILED DESCRIPTION

Using the imaging methods of the present disclosure, it may be desirable for a manufacturer or another to observe and/or analyze the surface of any of a wide range of consumer products. For example, it may be desirable to observe the surface of any product of interest, such as absorbent articles, cleaning products, batteries, beauty care products, medical products, medical devices, food products, and many other products. Absorbent articles for which a manufacturer may want to observe the surface include, for example, bath tissue, paper towels, wipes (e.g., disinfectant, cleansing, facial, hand, baby, perineal), diapers, training pants, catamenial products, wound care dressings, incontinence pads, adult incontinence pants, and bandages. Absorbent articles may be disposable absorbent articles. Cleaning products may include absorbent articles and may further include laundry products such as fabric softener sheets and dish care products such as cleaning foam. Beauty care products may include, for example, antiperspirants, toothpaste, skin care products such as lotion, cosmetics such as pressed powders, and hair care products such as hairspray. Medical products and medical devices may include medications, pharmaceuticals, bandages such as athletic bandages or wound care bandages, syrups, salves, etc. Food products may include, for example, snacks such as chips and crisps, liquids such as oils, emulsions such as margarine, beverages, etc. The example embodiments described herein may be used with any suitable consumer product, any portion or constituent of a consumer product, any of the above in packaging, etc.

Observation and analysis of the products as disclosed herein can provide a range of useful information, and it can do so in different contexts. For example, it may be desirable for various products to have a range of performance characteristics. Performance characteristics that may be relevant to certain products may include, by way of example only, the dimensions of the product, fit of the product in-use including determining the area(s) and amount of less than desirable fit, expansion of the product when in-use, contraction of the product when in use, how the product reacts in a static state, how the product reacts in a dynamic state, how the product reacts while being used, appearance of the product in-use including determining the location, position, coverage, and visibility of, for example, graphics, and many other factors that affect the product, its packaging, etc. An examination of the surface of the product may provide information regarding one or more of these characteristics.

Moreover, it may be desirable to observe the surface of various products in actual or simulated usage conditions. For purposes of illustration only, one example of a product category whose surface may be of interest during use conditions is absorbent articles. Illustrative absorbent articles include, but are not limited to, diapers, incontinence pads, incontinence pants, training pants, tampons, catamenial napkins, etc. Each of these examples may include an absorbent core that may receive and hold bodily exudates and/or a sealing member that mitigates the flow of exudates between the absorbent article and a wearer of the absorbent article. Any absorbent article may be evaluated according to the methods described herein.

One performance characteristic of absorbent articles that may be of interest is the product's ability to absorb and contain bodily discharges, including initial, subsequent, and continuous or repeated discharges. Another characteristic of absorbent articles that may be of interest to a manufacturer is the actual fit and appearance of the absorbent article during initial application and upon wear and loading, including repeated loading.

Methods for observing and analyzing the surface of a consumer product, and, more specifically, for observing and analyzing the surface of a consumer product, either as manufactured or as used, are provided. Disclosed are methods of observing the surface of product designs using techniques for obtaining data, such as imaging techniques. Any suitable surface imaging technique may be used with the present invention. Examples of suitable surface imaging processes include but are not limited to imaging using visible light and infrared light. Imaging using visible light may include, for example, white light, confocal or 3d laser scanning, structured or modulated light, stereoscopic and photometric systems, and stereo photogrammetry.

The methods described herein provide a means for evaluating performance characteristics of whole intact articles and/or articles in an in-use situation. Specifically, performance characteristics may be measured from an absorbent article and/or validated by data measured from an absorbent article.

As used herein, “absorbent article” refers to devices that absorb and contain body exudates, and, more specifically, refers to devices that can be placed against or in proximity to the body of a person to absorb and/or contain the various exudates discharged from the body. As used herein, the term “body fluids” or “body exudates” includes, but is not limited to, urine, blood, vaginal discharges, breast milk, sweat and fecal matter. Absorbent articles may include diapers, training pants, adult incontinence undergarments, feminine hygiene products, such as pads or tampons, breast pads, care mats, bibs, wound dressing products, and the like.

As used herein, “surface” refers to any portion of a product or panelist that is readily visible to the naked eye viewing the exterior of the product or panelist. In some examples, the surface of a product or panelist may include one or more of external surfaces and external components of the product or packaging.

“Disposable” is used in its ordinary sense to mean an article that is disposed or discarded after a limited number of usage events over varying lengths of time, for example, less than about 20 events, less than about 10 events, less than about 5 events, or less than about 2 events.

“Diaper” refers to an absorbent article generally worn by infants and incontinent persons about the lower torso so as to encircle the waist and legs of the wearer and that is specifically adapted to receive and contain urinary and fecal waste. As used herein, term “diaper” also includes “pants” which is defined below.

“Pant” or “training pant”, as used herein, refer to disposable garments having a waist opening and leg openings designed for infant or adult wearers. A pant may be placed in position on the wearer by inserting the wearer's legs into the leg openings and sliding the pant into position about a wearer's lower torso. A pant may be pre-formed by any suitable technique including, but not limited to, joining together portions of the article using refastenable and/or non-refastenable bonds (e.g., seam, weld, adhesive, cohesive bond, fastener, etc.). A pant may be preformed anywhere along the circumference of the article (e.g., side fastened, front waist fastened). While the terms “pant” or “pants” are used herein, pants are also commonly referred to as “closed diapers,” “prefastened diapers,” “pull-on diapers,” “training pants,” and “diaper-pants”. Suitable pants are disclosed in U.S. Pat. No. 5,246,433, issued to Hasse, et al. on Sep. 21, 1993; U.S. Pat. No. 5,569,234, issued to Buell, et al. on Oct. 29, 1996; U.S. Pat. No. 6,120,487, issued to Ashton on Sep. 19, 2000; U.S. Pat. No. 6,120,489, issued to Johnson, et al. on Sep. 19, 2000; U.S. Pat. No. 4,940,464, issued to Van Gompel, et al. on Jul. 10, 1990; U.S. Pat. No. 5,092,861, issued to Nomura, et al. on Mar. 3, 1992; U.S. Patent Publication No. 2003/0233082 A1, entitled “Highly Flexible And Low Deformation Fastening Device”, filed on Jun. 13, 2002; U.S. Pat. No. 5,897,545, issued to Kline, et al. on Apr. 27, 1999; U.S. Pat. No. 5,957,908, issued to Kline, et al on Sep. 28, 1999.

As used herein, “visible light” refers to the part of the electromagnetic spectrum that has wavelengths from about 390 to 700 nm.

As used herein, “infrared light” refers to infrared radiation ranges from the red end of the visible spectrum (around 700 nm) to wavelengths of about 1 mm.

As used herein, “pixel” refers to a basic unit of a two dimensional image or representation.

As used herein, “voxel” refers to a basic unit of a three dimensional image or representation.

As used herein, “texture map” refers to two dimensional images that are used to colorize a three dimensional surface representation.

As used herein, “panelist” refers to a human individual, such as an adult, toddler, or infant, or a mannequin. The mannequin may be indicative of an actual person and/or group of people (i.e., a composite of features from a group or statistical representation of a group of people). Specifically, as described by U.S. Patent Application No. 2008/0015537 assigned to The Procter & Gamble Company of Cincinnati, Ohio, the pertinent portions of which are incorporated by reference herein, a mannequin may be constructed to correspond to a suitable model infant (e.g., an infant weighing approximately 11.8 kilograms and had proportions typical for a median child in the recommended weight range for PAMPERS size 4 Easy Ups (7.3-15.4 kilograms)). It is to be appreciated that the mannequin can be of varying sizes to simulate varying human sizes such as, for example, an adult, a child, or an infant human, as well as male or female. This permits evaluation of different products for different users. The mannequin can be of any desired configuration and geometry to represent any given body size, shape, or composition. The mannequin may have a hollow interior portion, which may be adapted to receive a dispenser. Alternatively, the mannequin may comprise foam disposed in the interior portion.

As is noted above, performance characteristics may be measured and/or derived from actual physical testing. Performance characteristics include any measurable parameter related to the overall performance of the absorbent article such as fit of the article to a wearer and appearance of the article on the wearer. For example, performance characteristics pertaining to fit may include elastic components and their fit to the body, the behavior of absorbent article when dry/wet, fit of the product chassis on a wearer, article behavior during movement, etc.

Visible light or infrared light is used to produce images of a panelist or other consumer product. The images may be used to produce a three dimensional surface representation of the panelist and/or the consumer product. Multiple images may be collected to produce a three dimensional surface representation. As previously discussed, imaging using visible light or infrared light allows the panelist to include both human individuals and mannequins, also referred to herein as models. However, it is to be appreciated that imaging human individuals allows for a better understanding of how the consumer product, such as an absorbent article, will fit and appear in actual use.

For example, in some embodiments, where applicable, the product may be placed on a model so as to obtain information relating to how the product performs or responds under actual product usage conditions. The model may be one that represents, that simulates, etc. conditions for actual product usage. The model may have various features that enable the product to be used in a manner that simulates actual usage with respect to the information of interest. For example, where the product of interest is an absorbent article, such as a diaper, the model may take the form of a frame over which the diaper is positioned, or the model may take the form of a mannequin that represents the applicable anatomical geometry of the product's user. Where a mannequin is used, features that simulate actual product usage may include a tube and artificial orifice, or more than one of each, to enable transport of fluid, solid, or semi-solid materials from the inside of the mannequin to the absorbent article from an anatomically correct location, such as in a manner that represents soiling of a diaper by urine from a urethra, fecal matter from a rectum, or both. Specifically, the tube may be dimensioned, constructed, sized, and positioned to simulate a urethra and/or a rectum.

The mannequin may have a hollow interior portion, which may be adapted to receive a dispenser. The dispenser may carry either fluid and/or semi-solids to the mannequin. Alternatively, the dispenser or multiple dispensers, may be disposed within the mannequin. The mannequin may comprise more than one dispenser such that both urine and fecal incidents can be simulated. The fluids or semi-solids may exit the dispenser and mannequin through an opening in the mannequin. The product may be oriented such that it is adjacent the opening of the mannequin and may be adapted to receive the exited contents of the dispenser. The liquids and/or semi-solids may be moved through the mannequin in any desired manner. For example, the rate, quantity, consistency, frequency, etc. of fluid and/or semi-solid flow may be varied to represent any combination of parameters of interest. Doing so may provide information, for example, as to how well the article contains and distributes fluids when worn by a 10 pound boy child, or how well an article contains runny fecal material when worn by an incontinent two-hundred and twenty pound adult female, etc.

Similar to the above, the panelist may be a human individual of varying sizes to best simulate the use of the consumer product, such as an absorbent article. In the instance of observing and analyzing an absorbent article, the absorbent article may be prepared similar to the above. For example, the absorbent article may be prepared with fluid, semi-solid flow, and/or solid material to represent parameters of interest. Preparation of the absorbent article will be described in greater detail herein.

The methods discussed herein may be used to observe and analyze consumer products. While the present disclosure relates to observing and analyzing absorbent articles in the form of diapers, it is to be appreciated that the methods and apparatuses disclosed herein may be used with various types of absorbent articles and other consumer products.

The following description provides an overview of absorbent articles that may be observed and analyzed using the methods disclosed herein.

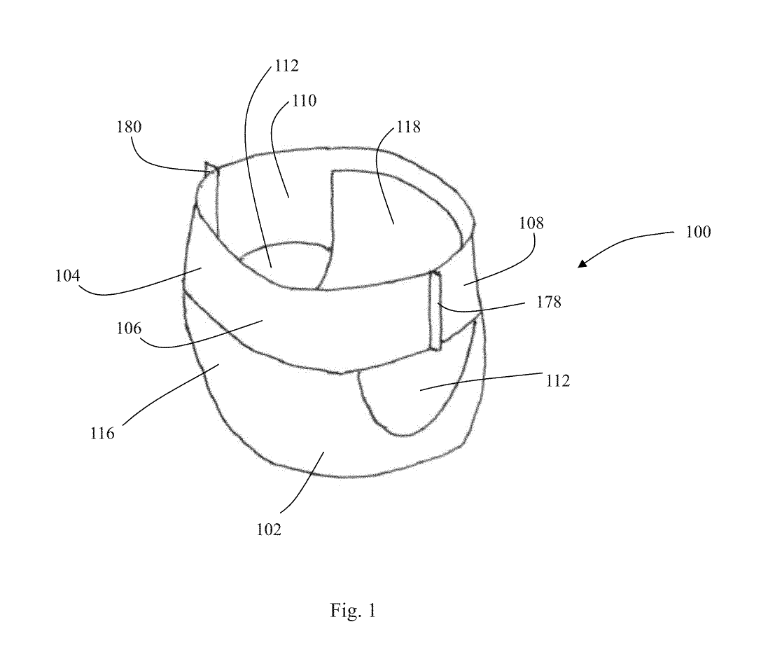

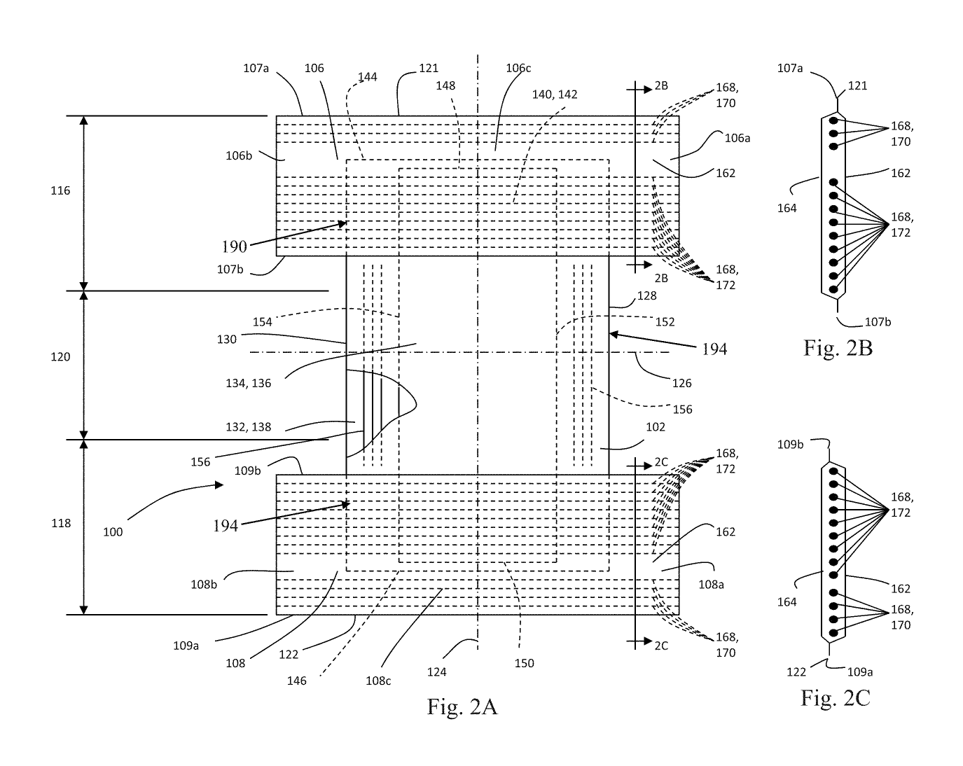

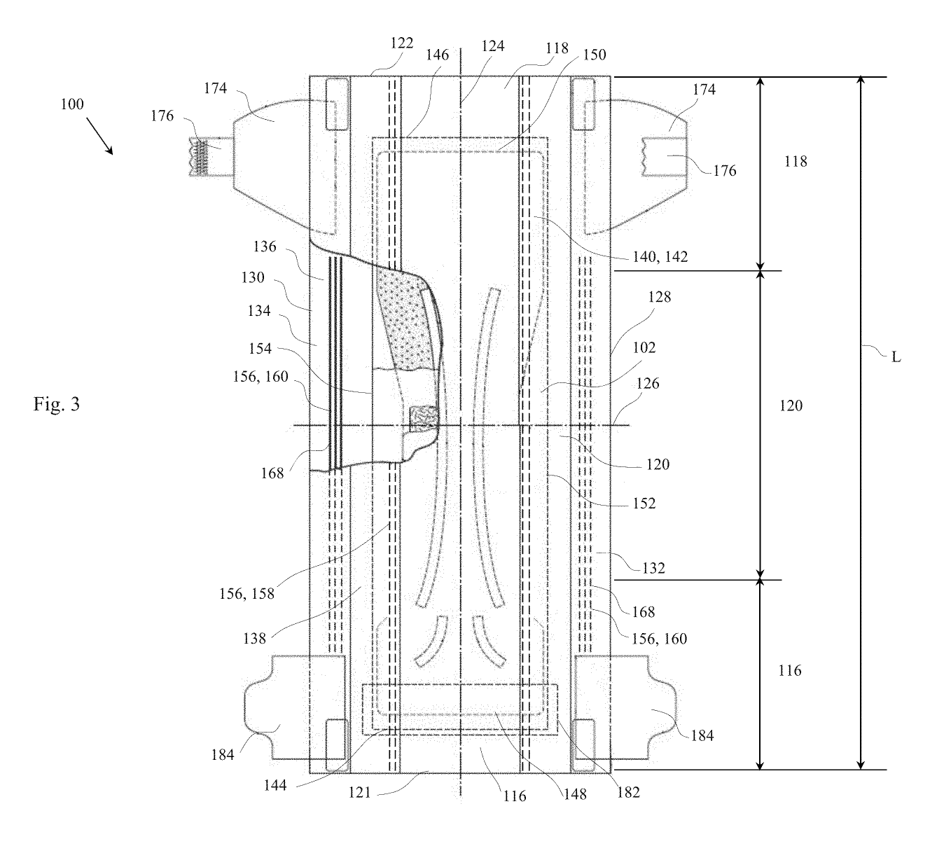

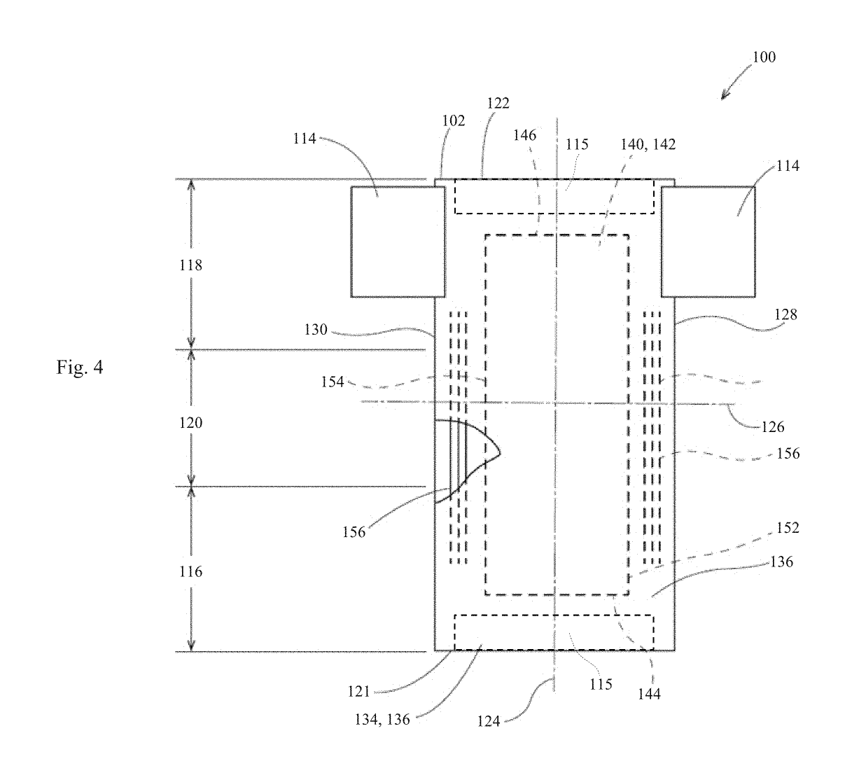

FIGS. 1, 2A, 3, 4, 5, and 6 illustrate an example of an absorbent article 100, such as a diaper, that may be analyzed with the methods discussed herein. In particular, FIG. 1 shows a perspective view of an absorbent article 100 in a pre-fastened configuration, and FIG. 2A shows a plan view of the absorbent article 100 with the portion of the diaper that faces away from a wearer oriented towards the viewer. The absorbent article 100 shown in FIGS. 1 and 2A includes a chassis 102 and a ring-like elastic belt 104. As discussed below in more detail, a first belt 106 and a second belt 108, which are both elastic, are connected together to form the ring-like elastic belt 104.

The chassis 102 includes a first waist region 116, a second waist region 118, and a crotch region 120 disposed intermediate the first and second waist regions. The first waist region 116 may be configured as a front waist region, and the second waist region 118 may be configured as back waist region. In some embodiments, the length of each of the front waist region, back waist region, and crotch region 120 may be 1/3 of the length of the absorbent article 100. The diaper 100 may also include a laterally extending front waist edge 121 in the front waist region 116 and a longitudinally opposing and laterally extending back waist edge 122 in the back waist region 118. To provide a frame of reference for the present discussion, the absorbent article 100 and chassis 102 is shown with a longitudinal axis 124 and a lateral axis 126. In some embodiments, the longitudinal axis 124 may extend through the front waist edge 121 and through the back waist edge 122. And the lateral axis 126 may extend through a first longitudinal or right side edge 128 and through a midpoint of a second longitudinal or left side edge 130 of the chassis 102.

The absorbent article 100 may include an inner, body facing surface 132, and an outer, garment facing surface 134. The chassis 102 may include a backsheet 136 and a topsheet 138. The chassis 102 may also include an absorbent assembly 140 including an absorbent core 142 that may be disposed between a portion of the topsheet 138 and the backsheet 136. As discussed in more detail below, the absorbent article 100 may also include other features, such as a waistband, leg elastics, and/or leg cuffs to enhance the fit around the legs of the wearer.

The periphery of the chassis 102 may be defined by the first longitudinal side edge 128, a second longitudinal side edge 130; a first laterally extending end edge 144 disposed in the first waist region 116; and a second laterally extending end edge 146 disposed in the second waist region 118. Both side edges 128 and 130 extend longitudinally between the first end edge 144 and the second end edge 146. When the absorbent article 100 is worn on the lower torso of a wearer, the front waist edge 121 and the back waist edge 122 of the chassis 102 may encircle a portion of the waist of the wearer. At the same time, the chassis side edges 128 and 130 may encircle at least a portion of the legs of the wearer. Moreover, the crotch region 120 may be generally positioned between the legs of the wearer with the absorbent core 142 extending from the front waist region 116 through the crotch region 120 to the back waist region 118. The chassis 102 may have opposing longitudinal edges that are oriented generally parallel to the longitudinal centerline 124. However, for better fit, longitudinal edges 128, 130 may be curved or angled to produce, for example, an “hourglass” shape diaper when viewed in a plan view, such as disclosed in U.S. Pat. No. 8,939,957 and U.S. Patent Publication No. 2012/0277702.

It is also to be appreciated that a portion or the whole of the absorbent article 100 may also be made laterally extensible. The additional extensibility may help allow the absorbent article 100 to conform to the body of a wearer during movement by the wearer. The additional extensibility may also help, for example, allow the diaper 100, including a chassis 102 having a particular size before extension, to extend in the front waist region 116, the back waist region 118, or both waist regions of the diaper 100 and/or chassis 102 to provide additional body coverage for wearers of differing size, i.e., to tailor the diaper to an individual wearer. Such extension of the waist region or regions may give the absorbent article a generally hourglass shape, so long as the crotch region is extended to a relatively lesser degree than the waist region or regions, and may impart a tailored appearance to the article when it is worn.

As previously mentioned, the diaper 100 may include a backsheet 136. The backsheet 136 may also define the outer surface 134 of the chassis 102. The backsheet 136 may be impervious or at least partially impervious to fluids (e.g., menses, urine, and/or runny feces) and may be manufactured from a thin plastic film, although other flexible liquid impervious materials may also be used. The backsheet 136 may prevent the exudates absorbed and contained in the absorbent core from wetting articles that contact the diaper 100, such as bedsheets, clothes, and undergarments. The backsheet 136 may also include a woven or nonwoven material, polymeric films such as thermoplastic films of polyethylene or polypropylene, and/or a multi-layer or composite materials comprising a film and a nonwoven material (e.g., having an inner film layer and an outer nonwoven layer). The backsheet may also include an elastomeric film. An example backsheet 136 may be a polyethylene film having a thickness of from about 0.012 mm (0.5 mils) to about 0.051 mm (2.0 mils). Exemplary polyethylene films are manufactured by Clopay Corporation of Cincinnati, Ohio, under the designation BR-120 and BR-121 and by Tredegar Film Products of Terre Haute, Ind., under the designation XP-39385. The backsheet 136 may also be embossed and/or matte-finished to provide a more clothlike appearance. Further, the backsheet 136 may permit vapors to escape from the absorbent core (i.e., the backsheet is breathable) while still preventing exudates from passing through the backsheet 136. The size of the backsheet 136 may be dictated by the size of the absorbent core 142 and/or particular configuration or size of the diaper 100.

In one embodiment, an adhesive may be applied to the garment-facing exterior of the backsheet for the purpose of holding the absorbent article in place by adhering to the wearer's underwear. Such adhesive may be especially desirable for use with adult incontinence and feminine hygiene type absorbent articles.

Also described above, the absorbent article 100 may include a topsheet 138. The topsheet 138 may also define all or part of the inner surface 132 of the chassis 102. The topsheet 138 may be compliant, soft feeling, and non-irritating to the wearer's skin. It may be elastically stretchable in one or two directions. Further, the topsheet 138 may be liquid pervious, permitting liquids (e.g., menses, urine, and/or runny feces) to penetrate through its thickness. A topsheet 138 may be manufactured from a wide range of materials such as woven and nonwoven materials; apertured or hydroformed thermoplastic films; apertured nonwovens, porous foams; reticulated foams; reticulated thermoplastic films; and thermoplastic scrims. Woven and nonwoven materials may comprise natural fibers such as wood or cotton fibers; synthetic fibers such as polyester, polypropylene, or polyethylene fibers; or combinations thereof. If the topsheet 138 includes fibers, the fibers may be spunbond, carded, wet-laid, meltblown, hydroentangled, or otherwise processed as is known in the art.

Topsheets 138 may be selected from high loft nonwoven topsheets, apertured film topsheets, and apertured nonwoven topsheets. Apertured film topsheets may be pervious to bodily exudates, yet substantially non-absorbent, and have a reduced tendency to allow fluids to pass back through and rewet the wearer's skin. Exemplary apertured films may include those described in U.S. Pat. Nos. 5,628,097; 5,916,661; 6,545,197; and 6,107,539.

In some embodiments, the topsheet may comprise graphics such that depth perception is created as described in U.S. Pat. No. 7,163,528.

The absorbent article 100 may also include an absorbent assembly 140 that is joined to the chassis 102. The absorbent assembly 140 may have a laterally extending front edge 148 in the front waist region 116 and may have a longitudinally opposing and laterally extending back edge 150 in the back waist region 118. The absorbent assembly may have a longitudinally extending right side edge 152 and may have a laterally opposing and longitudinally extending left side edge 154, both absorbent assembly side edges 152 and 154 may extend longitudinally between the front edge 148 and the back edge 150. The absorbent assembly 140 may additionally include one or more absorbent cores 142 or absorbent core layers. The absorbent core 142 may be at least partially disposed between the topsheet 138 and the backsheet 136 and may be formed in various sizes and shapes that are compatible with the diaper. Exemplary absorbent structures for use as the absorbent core of the present disclosure are described in U.S. Pat. Nos. 4,610,678; 4,673,402; 4,888,231; and 4,834,735.

Some absorbent core embodiments may comprise fluid storage cores that contain reduced amounts of cellulosic airfelt material. For instance, such cores may comprise less than about 40%, 30%, 20%, 10%, 5%, or even 1% of cellulosic airfelt material. Such a core may comprise primarily absorbent gelling material in amounts of at least about 60%, 70%, 80%, 85%, 90%, 95%, or even about 100%, where the remainder of the core may comprise a microfiber glue (if applicable). Such cores, microfiber glues, and absorbent gelling materials are described in U.S. Pat. Nos. 5,599,335; 5,562,646; 5,669,894; and 6,790,798 as well as U.S. Patent Publication Nos. 2004/0158212 and 2004/0097895.

The absorbent article 100 may also include elasticized leg cuffs 156. It is to be appreciated that the leg cuffs 156 may be and are sometimes also referred to as leg bands, side flaps, barrier cuffs, elastic cuffs, or gasketing cuffs. The elasticized leg cuffs 156 may be configured in various ways to help reduce the leakage of body exudates in the leg regions. For example, in some embodiments, a gasketing leg cuff 160 may be positioned adjacent to the side edge 130, 128 of the chassis 102 and a barrier leg cuff 158 may be positioned between a gasketing leg cuff 160 and the longitudinal axis 124 of the absorbent article 100. Example leg cuffs 156 may include those described in U.S. Pat. Nos. 3,860,003; 4,909,803; 4,695,278; 4,795,454; 4,704,115; 4,909,803; 9,089,455 and U.S. Patent Publication Nos. 2009/0312730; 2012/0330262; 2012/0330263; 2012/0330264; and 2013/0255865.

As mentioned above, diaper pants may be manufactured with a ring-like elastic belt 104 and provided to consumers in a configuration wherein the front waist region 116 and the back waist region 118 are connected to each other as packaged, prior to being applied to the wearer. As such, the absorbent article may have a continuous perimeter waist opening 110 and continuous perimeter leg openings 112 such as shown in FIG. 1. As previously mentioned, the ring-like elastic belt 104 is defined by a first elastic belt 106 connected with a second elastic belt 108. As shown in FIG. 2A, the first elastic belt 106 defines first and second opposing end regions 106a, 106b and a central region 106c, and the second elastic 108 belt defines first and second opposing end regions 108a, 108b and a central region 108c.

The central region 106c of the first elastic belt is connected with the first waist region 116 of the chassis 102, and the central region 108c of the second elastic belt 108 is connected with the second waist region 118 of the chassis 102. As shown in FIG. 1, a portion of the first end region 106a of the first elastic belt 106 is connected with a portion of the first end region 108a of the second elastic belt 108 at first side seam 178, and a portion of the second end region 106b of the first elastic belt 106 is connected with a portion of the second end region 108b of the second elastic belt 108 at second side seam 180 to define the ring-like elastic belt 104 as well as the waist opening 110 and leg openings 112.

As shown in FIGS. 2A, 2B, and 2C, the first elastic belt 106 also defines an outer lateral edge 107a and an inner lateral edge 107b, and the second elastic belt 108 defines an outer lateral edge 109a and an inner lateral edge 109b. The outer lateral edges 107a, 109a may also define the front waist edge 121 and the laterally extending back waist edge 122. The first elastic belt and the second elastic belt may also each include an outer, garment facing layer 162 and an inner, wearer facing layer 164. It is to be appreciated that the first elastic belt 106 and the second elastic belt 108 may comprise the same materials and/or may have the same structure. In some embodiments, the first elastic belt 106 and the second elastic belt may comprise different materials and/or may have different structures. It should also be appreciated that the first elastic belt 106 and the second elastic belt 108 may be constructed from various materials. For example, the first and second belts may be manufactured from materials such as plastic films; apertured plastic films; discrete strands; woven or nonwoven webs of natural materials (e.g., wood or cotton fibers), synthetic fibers (e.g., polyolefins, polyamides, polyester, polyethylene, or polypropylene fibers) or a combination of natural and/or synthetic fibers; or coated woven or nonwoven webs. In some embodiments, the first and second elastic belts may include a nonwoven web of synthetic fibers, and may include a stretchable nonwoven. In other embodiments, the first and second elastic belts may include an inner hydrophobic, non-stretchable nonwoven material and an outer hydrophobic, non-stretchable nonwoven material.

The first and second elastic belts 106, 108 may also each include belt elastic material interposed between the outer layer 162 and the inner layer 164. The belt elastic material may include one or more elastic elements such as strands, ribbons, or panels extending along the lengths of the elastic belts. As shown in FIGS. 2A, 2B, and 2C, the belt elastic material may include a plurality of elastic strands 168 that may be referred to herein as outer, waist elastics 170 and inner, waist elastics 172.

As shown in FIG. 2A, the outer, waist elastics 170 extend continuously laterally into and between the first and second opposing end regions 106a, 106b and across the central region 106c of the first elastic belt 106 and into and between the first and second opposing end regions 108a, 108b and across the central region 108c of the second elastic belt 108. In some embodiments, some elastic strands 168 may be configured with discontinuities in areas. For example, as shown in FIG. 2, the inner, waist elastics 172 extend intermittently along the first and second elastic belts 106, 108. More particularly, the inner, waist elastics 172 extend along the first and second opposing end regions 106a, 106b and partially across the central region 106c of the first elastic belt 106. The inner, waist elastics 172 also extend along the first and second opposing end regions 108a, 108b and partially across the central region 108c of the second elastic belt 108. As such, the inner, waist elastics 172 may not extend across the entirety of the central regions 106c, 108c of the first and second elastic belts 106, 108. Thus, for example, some elastic strands 168 may not extend continuously through regions of the first and second elastic belts 106, 108 where the first and second elastic belts 106, 108 overlap the absorbent assembly 140. In some embodiments, some elastic strands 168 may partially extend into regions of the first and second elastic belts 106, 108 where the first and second elastic belts 106, 108 overlap the absorbent assembly 140. In some embodiments, some elastic strands 168 may not extend into any region of the first and second elastic belts 106, 108 where the first and second elastic belts 106, 108 overlap the absorbent assembly 140. It is to be appreciated that the first and/or second elastic belts 106, 108 may be configured with various configurations of discontinuities in the outer, waist elastics 170 and/or the inner, waist elastic elastics 172.

In some embodiments, the elastic strands 168 may be disposed at a constant interval in the longitudinal direction. In other embodiments, the elastic strands 168 may be disposed at different intervals in the longitudinal direction. As discussed in more detail below, the belt elastic strands 168, in a stretched condition, may be interposed and joined between the uncontracted outer layer and the uncontracted inner layer. When the belt elastic material is relaxed, the belt elastic material returns to an unstretched condition and contracts the outer layer and the inner layer. The belt elastic material may provide a desired variation of contraction force in the area of the ring-like elastic belt. It is to be appreciated that the chassis 102 and elastic belts 106, 108 may be configured in different ways other than as depicted in FIG. 2A.

In some embodiments, the absorbent article 100 may comprise front ears 184 and back ears 174. The front ears 184 and the back ears 174 may be an integral part of the chassis 102. For example, the front ears 184 and the back ears 174 may be formed from the topsheet 138 and/or the backsheet 136. Alternatively, the front ears 184 and the back ears 174 may be attached to the backsheet 136 and/or the topsheet 138. The front ears 184 and the back ears 174 may be extensible to facilitate attachment on the landing zone 182 and to maintain placement around the waist of the wearer. The back ears 174 may comprise a tab member 176. The tab member 176 may be attached to a portion of the back ears 174 to facilitate attachment to the landing zone 182.

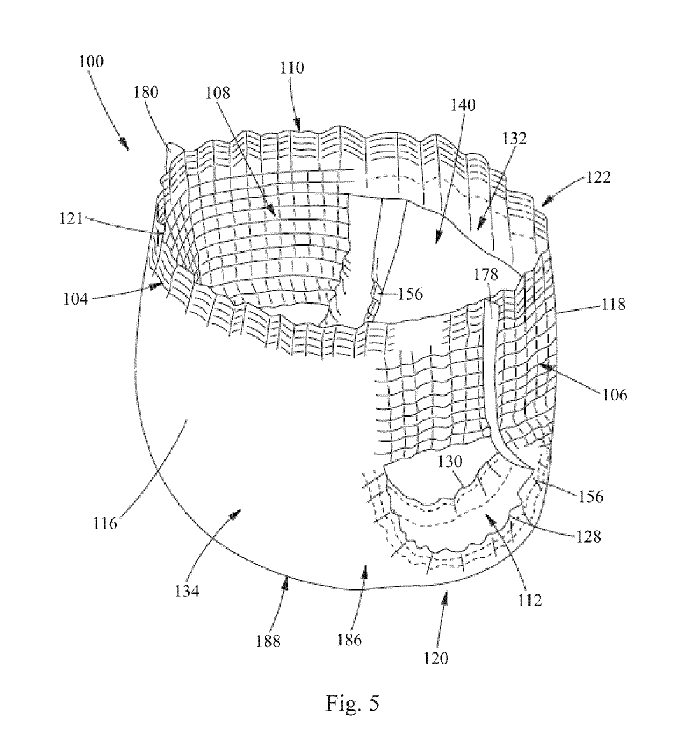

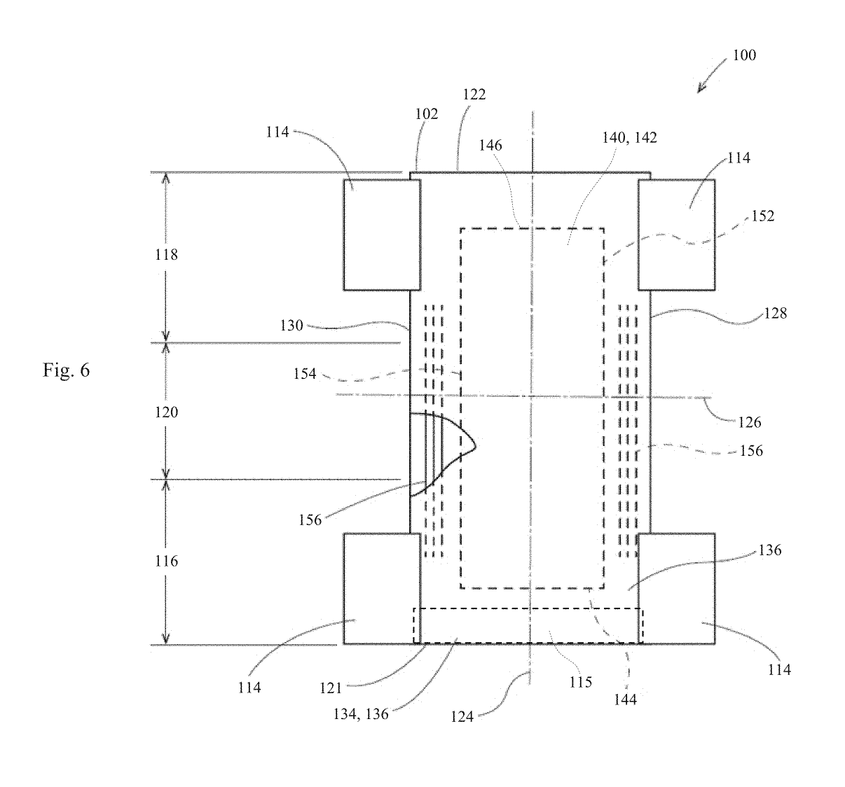

In some embodiment, referring to FIGS. 5 and 6, the article 100 may comprise an elasticized waistband 115. The elasticized waistband may provide improved fit and containment and may be configured to elastically expand and contract laterally to dynamically fit a wearer's waist. The elasticized waistband may extend longitudinally outwardly from the waist edge of the absorbent article 100 toward the edge of the absorbent core 142. In one embodiment, the absorbent article 100 may have two elasticized waistbands, one positioned in the back waist region 118 and one positioned in the front waist region 116, although other embodiments may be constructed with a single elasticized waistband. The elasticized waistband may be constructed in a number of different configurations including those described in U.S. Pat. Nos. 4,515,595 and 5,151,092. Further, the waistband may be constructed as disclosed in U.S. Publication Nos. 2012/0330262; 2012/0330263; and 2012/0330264 such that the waistband works in combination with the leg cuffs to provide improved fit and containment.

In some embodiments, the elasticized waistbands may comprise materials that have been “prestrained” or “mechanically prestrained” (i.e., subjected to some degree of localized pattern mechanical stretching to permanently elongate the material). In some embodiments, the materials may be prestrained using suitable deep embossing techniques. In other embodiments, the materials may be prestrained by directing the material through an incremental mechanical stretching system as described in U.S. Pat. No. 5,330,458. The materials may then be allowed to return to their substantially untensioned condition, thus forming a zero strain stretch material that is extensible, at least up to the point of initial stretching. Examples of zero strain materials are disclosed in U.S. Pat. Nos. 2,075,189; 3,025,199; 4,107,364; 4,209,563; 4,834,741; and 5,151,092. The waistband may be any shape and size that allows the absorbent article to fit the wearer as desired about the waist region.

In some embodiments, the waistband may be positioned between the side panels 114 and/or the back ears 174 and/or front ears 184. In other embodiments the waistband may be positioned such that a portion of the waistband overlaps a portion of the side panels 114 and/or the back ears 174 and/or the front ears 184.

In some embodiments, the absorbent article 100 may comprise side panels 115. The side panels 115 may be discrete from or integral with the chassis 100. A discrete side panel is formed as a separate element that is joined to the chassis 100. In some embodiments, this includes a plurality of side panels, e.g. FIG. 5 or 6 (also referred to as ear panels or side flaps) being joined to the side edges 128, 130 of the chassis in the front and/or rear waist regions 118 and 116. The side panel may be attached to the garment facing surface 132, the body facing surface 132, or between the garment facing surface 132 and the body facing surface 132, such as between the topsheet 138 and the backsheet 136. In some embodiments, the waistbands 112 can overlap the side panels to create a continuous belt-like structure (not shown).

In some embodiments, the side panels in the back waist region may connect with the garment facing surface of the absorbent article in the front waist region to form a waist circumference that may encircle the wearer during wear of the absorbent article. In other embodiments, the side panels disposed in the back waist region may connect with the side panels disposed in the front waist region at a seam, which forms a waist circumference that may encircle the wearer during wear of the absorbent article. The seam may be an overlapping seam or a butt seam. Further, in some embodiments, the seam may be refastenable, such that the side panels may be detached and reattached, or permanent, such that the seam may not be detached and reattached.

The side panels may comprise an inner nonwoven layer and an outer nonwoven layer and elastic elements, such as elastic strands or a film, therebetween. The inner and outer nonwoven layers may be joined using adhesive or thermoplastic bonds. Various suitable side panel configurations can be found in U.S. Pub. No. 2013/0211363.

An integral side panel is a portion, one or more layers, of the chassis that projects laterally outward from the longitudinal edge. The integral flap may be formed by cutting the chassis to include the shape of the flap projection.

While many of the embodiments illustrated in this application having belt-like side flaps are pant articles, taped articles may have belt-like side flaps disposed in one or both waist regions as well. The side panels may be any shape that allows the absorbent article to fit the wearer as desired about the waist region and the leg openings.

The absorbent article may also include a fastening system. When fastened, the fastening system interconnects the front waist region 116 and the rear waist region 118 resulting in a waist circumference that may encircle the wearer during wear of the absorbent article 10. This may be accomplished by ears 174, 184 or side panels 115, for example. The ears 174 or side panels 115 in the back waist region interconnect with ears 184 or side panels 115 in the front waist region or by the flaps or side panels in the back waist region interconnecting with the chassis 100 in the front waist region. The fastening system may comprises a fastener 176 such as tape tabs, hook and loop fastening components, interlocking fasteners such as tabs and slots, buckles, buttons, snaps, and/or hermaphroditic fastening components, although any other known fastening means are generally acceptable. The fasteners may releasably engage with a landing zone 182, which may be a woven or nonwoven. Some exemplary surface fastening systems are disclosed in U.S. Pat. Nos. 3,848,594; 4,662,875; 4,846,815; 4,894,060; 4,946,527; 5,151,092; and 5,221,274. An exemplary interlocking fastening system is disclosed in U.S. Pat. No. 6,432,098. The fastening system may also provide a means for holding the article in a disposal configuration as disclosed in U.S. Pat. No. 4,963,140. The fastening system may also include primary and secondary fastening systems, as disclosed in U.S. Pat. No. 4,699,622. The fastening system may be constructed to reduce shifting of overlapped portions or to improve fit as disclosed in U.S. Pat. Nos. 5,242,436; 5,499,978; 5,507,736; and 5,591,152.

In some embodiments, such as illustrated in FIG. 5, the absorbent article 100, which may be referred to herein as a diaper pant, comprises an absorbent main body 188, also referred to herein as chassis 102, to cover the crotch region of the wearer and a belt 104 extending transversely about the waist opening 110. The absorbent article 100 may also comprise an outer cover layer 186 to cover the main body 188. The belt 104 defines the waist opening 110. The belt 104, the main body 188 and/or the outer cover layer 186 jointly define the leg opening 112. One or more of the belt layers may extend from a first waist edge 121 in a first waist region 116 through the crotch region to a longitudinally opposing second waist edge 122 in a second waist region 118 and may form a portion or the whole of the outer surface of the absorbent article 100.

The absorbent main body 188, also referred to as a chassis 102, absorbs and contains body exudates disposed on the main body 188. In the embodiment shown in FIG. 2A, the main body 188 has a generally rectangular shape having a longitudinal centerline 124, a transverse centerline 126, left and right longitudinally extending side edges 128, 130 and front and back transversely extending end edges 144, 146. The main body 188 also has waist panels (i.e., a front waist panel 190 positioned in the front waist region 116 of the absorbent article 100 and a back waist panel 192 positioned in the back waist region 118) and a crotch panel 194 in the crotch region 30 between the front and back waist panels 190, 192.

The absorbent articles 100 may comprise first and second belts 106, 108 intended to encircle at least a portion of the waist of the wearer, the first and second belt portions 106, 108 being connected by a main body 188 forming the crotch region 120 of the absorbent article 100. The first and second belts 106 and 108 may be formed from a first belt layer forming a portion of the outer surface of the absorbent article, the first belt layer 106 may be formed of two longitudinally spaced webs of material. The first and second belts 106 and 108 may also comprise a second belt layer forming a portion of the inner surface of the absorbent article 100, the second belt layer may also be formed of two longitudinally spaced webs of material. The second belt layer may also be discontinuous and spaced apart in a transverse direction. The first and second belt layers may be formed of substantially the same material or may comprise different materials. The first and second belt layers may be formed from nonwovens, films, foams, elastic nonwoven, or combinations thereof. The first and second belts 106, 108 may also comprise an elastomeric material disposed between the first and second belt layers. The elastomeric material may comprise one or more elastic strands, elastomeric films, elastomeric ribbons, elastomeric nonwovens, elastomeric filaments, elastomeric adhesives, elastomeric foams, scrims or combinations thereof. A portion of the elastomeric material may be directly combined with the outer cover layer 186. The first and second belts 106, 108 may overlap at least a portion of the main body and one or both of the belt portions may be disposed on the outer surface of the main body or alternatively on the inner surface of the main body. A portion of the second belt layer and/or a portion of the first belt layer may be directly attached to the outer cover layer 186. Alternatively, the first belt and second belt 106, 108 may comprise longitudinally spaced webs of material forming a first surface of the belt wherein the webs are folded along the waist edge, or alternatively the leg opening edge, of the belt to wrap the elastomeric material and form at least a portion of the second surface of the belt. In other words, at least a portion of the inner surface and outer surface of each of the belt portions may be formed from a single web of material. It is to be appreciated that the outer cover layer 186, main body 188, and first and second belts 106, 108 may be configured in a number of ways such as disclosed in U.S. Pat. No. 9,072,632.

It is to be appreciated that any of the aforementioned components of the absorbent article may include one or more substrates. For example, a substrate having a single layer with a basis weight from about 8 to about 40 g/m² may form a component or portion of a component of the absorbent article. It is also to be appreciated that the substrate may include more than one layer.

The methods of the disclosed may be used with any suitable feminine hygiene article as well. A feminine hygiene article may include a topsheet, backsheet, absorbent core, and other components as are known in the art.

The topsheet may be manufactured from a wide range of materials such as woven and nonwoven materials; polymeric materials such as apertured formed thermoplastic films, apertured plastic films, and hydroformed thermoplastic films; porous foams; reticulated foams; reticulated thermoplastic films; and thermoplastic scrims. Suitable woven and nonwoven materials can be included of natural fibers (e.g., wood or cotton fibers), synthetic fibers (e.g., polymeric fibers such as polyester, polypropylene, or polyethylene fibers) or from a combination of natural and synthetic fibers. In one embodiment, the topsheet may be made of a hydrophobic material to isolate the wearer's skin from liquids which have passed through the topsheet. If the topsheet is made of a hydrophobic material, at least the upper surface of the topsheet is treated to be hydrophilic so that liquids will transfer through the topsheet more rapidly. This diminishes the likelihood that body exudates will flow off the topsheet rather than being drawn through the topsheet and being absorbed by the absorbent core.

The topsheet can include an apertured formed film. Apertured formed films can be used for the topsheet because they are pervious to body exudates and yet non-absorbent and have a reduced tendency to allow liquids to pass back through and rewet the wearer's skin. Thus, the surface of the formed film which is in contact with the body remains dry, thereby reducing body soiling and creating a more comfortable feel for the wearer. Suitable formed films are described in U.S. Pat. No. 3,929,135, entitled “Absorptive Structures Having Tapered Capillaries”, issued to Thompson on Dec. 30, 1975; U.S. Pat. No. 4,324,246 entitled “Disposable Absorbent Article Having A Stain Resistant Topsheet”, issued to Mullane, et al. on Apr. 13, 1982; U.S. Pat. No. 4,342,314 entitled “Resilient Plastic Web Exhibiting Fiber-Like Properties”, issued to Radel, et al. on Aug. 3, 1982; U.S. Pat. No. 4,463,045 entitled “Macroscopically Expanded Three-Dimensional Plastic Web Exhibiting Non-Glossy Visible Surface and Cloth-Like Tactile Impression”, issued to Ahr, et al. on Jul. 31, 1984; and U.S. Pat. No. 5,006,394 “Multilayer Polymeric Film” issued to Baird on Apr. 9, 1991.

The absorbent core can be any absorbent member which is generally compressible, conformable, non-irritating to the wearer's skin, and capable of absorbing and retaining body fluids. The absorbent core may be manufactured in a wide variety of sizes and shapes (e.g., rectangular, hourglass, “T” -shaped, asymmetric, etc.) and from a wide variety of liquid-absorbent materials commonly used in disposable pull-on garments and other absorbent articles such as comminuted wood pulp which is generally referred to as airfelt. Examples of other suitable absorbent materials include creped cellulose wadding; meltblown polymers including coform; chemically stiffened, modified or cross-linked cellulosic fibers; tissue including tissue wraps and tissue laminates; absorbent foams; absorbent sponges; superabsorbent polymers; absorbent gelling materials; or any equivalent material or combinations of materials.

The configuration and construction of the absorbent core may vary (e.g., the absorbent core may have varying caliper zones, a hydrophilic gradient, a superabsorbent gradient, or lower average density and lower average basis weight acquisition zones; or may include one or more layers or structures). Further, the size and absorbent capacity of the absorbent core may also be varied to accommodate wearers ranging from infants through adults. However, the total absorbent capacity of the absorbent core should be compatible with the design loading and the intended use of the absorbent article.

The absorbent core structure may comprise a heterogeneous mass layer as those described in US patent application No. 61/988,565, filed May 5, 2014; US patent application No. 62/115,921, filed Feb. 13, 2015; or US patent application No. 62/018,212. The heterogeneous mass layer has a depth, a width, and a height. The heterogeneous mass may have high internal phase emulsion (HIPE) foam. One method for forming HIPEs using a continuous process is described in U.S. Pat. No. 5,149,720 (DesMarais, et al), issued Sep. 22, 1992; U.S. Pat. No. 5,827,909 (DesMarais) issued Oct. 27, 1998; and U.S. Pat. No. 6,369,121 (Catalfamo, et al.) issued Apr. 9, 2002.

The absorbent core structure may comprise a substrate and superabsorbent polymer layer as those described in U.S. Pat. No. 8,124,827 filed on Dec. 2, 2008 (Tamburro); U.S. application Ser. No. 12/718,244 published on Sep. 9, 2010; U.S. application Ser. No. 12/754,935 published on Oct. 14, 2010; or U.S. Pat. No. 8,674,169 issued on Mar. 18, 2014.

The absorbent core may include other optional components. One such optional component is the core wrap, i.e., a material, typically but not always a nonwoven material, which either partially or totally surrounds the core. Suitable core wrap materials include, but are not limited to, cellulose, hydrophilically modified nonwoven materials, perforated films and combinations thereof.

The backsheet can comprise a liquid impervious film. The backsheet can be impervious to liquids (e.g., body fluids) and can be typically manufactured from a thin plastic film. However, typically the backsheet can permit vapours to escape from the disposable article. In an embodiment, a microporous polyethylene film can be used for the backsheet. A suitable microporous polyethylene film is manufactured by Mitsui Toatsu Chemicals, Inc., Nagoya, Japan and marketed in the trade as PG-P.

One suitable material for the backsheet can be a liquid impervious thermoplastic film having a thickness of from about 0.012 mm (0.50 mil) to about 0.051 mm (2.0 mils), for example including polyethylene or polypropylene. Typically, the backsheet can have a basis weight of from about 5 g/m² to about 35 g/m². However, it should be noted that other flexible liquid impervious materials may be used as the backsheet. Herein, “flexible” refers to materials which are compliant and which will readily conform to the general shape and contours of the wearer's body.

The backsheet can be typically positioned adjacent the outer-facing surface of the absorbent core and can be joined thereto by any suitable attachment device known in the art. For example, the backsheet may be secured to the absorbent core by an adhesive. An example of a suitable attachment device including an open pattern network of filaments of adhesive is disclosed in U.S. Pat. No. 4,573,986 entitled “Disposable Waste-Containment Garment”, which issued to Minetola, et al. on Mar. 4, 1986. Another suitable attachment device including several lines of adhesive filaments swirled into a spiral pattern is illustrated by the apparatus and methods shown in U.S. Pat. No. 3,911,173 issued to Sprague, Jr. on Oct. 7, 1975; U.S. Pat. No. 4,785,996 issued to Ziecker, et al. on Nov. 22, 1978; and U.S. Pat. No. 4,842,666 issued to Werenicz on Jun. 27, 1989. Alternatively, the attachment device may include heat bonds, pressure bonds, ultrasonic bonds, dynamic mechanical bonds, or any other suitable attachment device or combinations of these attachment devices.

The backsheet may be additionally secured to the topsheet by any of the above-cited attachment devices. The topsheet, the backsheet, and the absorbent core can be assembled in a variety of well- known configurations, including so called “tube” products or side flap products, such as, for example, configurations are described generally in U.S. Pat. No. 4,950,264, “Thin, Flexible Sanitary Napkin” issued to Osborn on Aug. 21, 1990, U.S. Pat. No. 4,425,130, “Compound Sanitary Napkin” issued to DesMarais on Jan. 10, 1984; U.S. Pat. No. 4,321,924, “Bordered Disposable Absorbent Article” issued to Ahr on Mar. 30, 1982; U.S. Pat. No. 4,589,876, and “Shaped Sanitary Napkin With Flaps” issued to Van Tilburg on Aug. 18, 1987. Each of these patents is incorporated herein by reference.

The absorbent article may also include such other suitable components as are known in the art including, but not limited to, re-closable fastening system, lotion, acquisition layers, distribution layers, wetness indicators, sensors, elasticized waist bands and other similar additional elastic elements and the like, belts and the like, waist cap features, containment and aesthetic characteristics and combinations thereof. For example, the absorbent article, such as a feminine hygiene product, may comprise “wings” (not shown) intended to wrap the edges of the wearer's undergarments in the crotch region and/or affix the article to the undergarment to avoid poor folding and premature detachment. Exemplary absorbent articles comprising wings are disclosed in U.S. Pat. No. 8,039,685.

It is to be appreciated that the features of the absorbent article described herein may be excluded or combined to form various embodiments of an absorbent article. Although the following methods may be provided in the context of absorbent articles 100, it is to be appreciated that the methods herein may be used to observe and analyze absorbent articles, such as for example, disclosed in U.S. Pat. Nos. 7,569,039 and 9,072,632; U.S. Patent Publication Nos. 2005/0107764 A1, 2012/0061016 A1, and 2012/0061015 A1; 2013/0255861 A1; 2013/0255862 A1; 2013/0255863 A1; 2013/0255864 A1; and 2013/0255865 A1; and U.S. Patent Application Ser. Nos. 62/136,003 filed on Mar. 20, 2015 entitled “DISPOSABLE ABSORBENT ARTICLES AND ARRAYS OF SAID ARTICLES COMPRISING VISUAL CHARACTERISTICS”; U.S. Pat. No. 14/996,683 filed on Jan. 15, 2016 entitled “ADULT DISPOSABLE ABSORBENT ARTICLES AND ARRAYS OF SAID ARTICLES COMPRISING ABSORBENT CORES HAVING CHANNELS”; and 62/286,662 filed on Jan. 25, 2016 entitled “ABSORBENT ARTICLES COMPRISING SPACERS.”

As previously mentioned, the methods according to the present disclosure may be used to analyze the surface of consumer products, such as absorbent article. More specifically, the methods of the present disclosure may be used to analyze and compare consumer products to obtain information on the fit and/or appearance of the consumer products prior to, during, and after use.

Imaging the surface of a panelist using visible light and/or infrared light avoids the problems previously discussed, such as exposure to ionizing radiation, exposure to restrictive, noisy environments, and relatively high cost. However, by having only a single three dimensional surface representation there may not be enough information to obtain the desired result. For example, by having a single three dimensional surface representation of a single panelist wearing an absorbent article, the absorbent article may hide features of the panelist that would be helpful to locate, such as the lowest point of the crotch region of the panelist. Further, having a single three dimensional surface representation does not allow for comparison of the absorbent article after the absorbent article has undergone preparing. Thus, there is no reference to, for example, how the fit or appearance of the absorbent article has changed, or how the absorbent article fits or appears with reference to features of the panelist.

Thus, the present disclosure is related to a method including obtaining at least two three dimensional surface representations, such as a first three dimensional surface representation and a second three dimensional surface representation that may be compared to obtain fit and appearance information. For example, in some embodiments, a first three dimensional surface representation may be obtained and a second three dimensional surface representation may be obtained. The first and second three dimensional surface representations may be overlapped with one another to obtain relevant fit and appearance data, such as, for example, a difference in volume and surface area. Thus, the methods disclosed herein allow for a panelist that is a human individual to be imaged and for relevant fit and appearance data to be obtained based on the images and generated three dimensional surface representations. These methods will be discussed in detail herein.

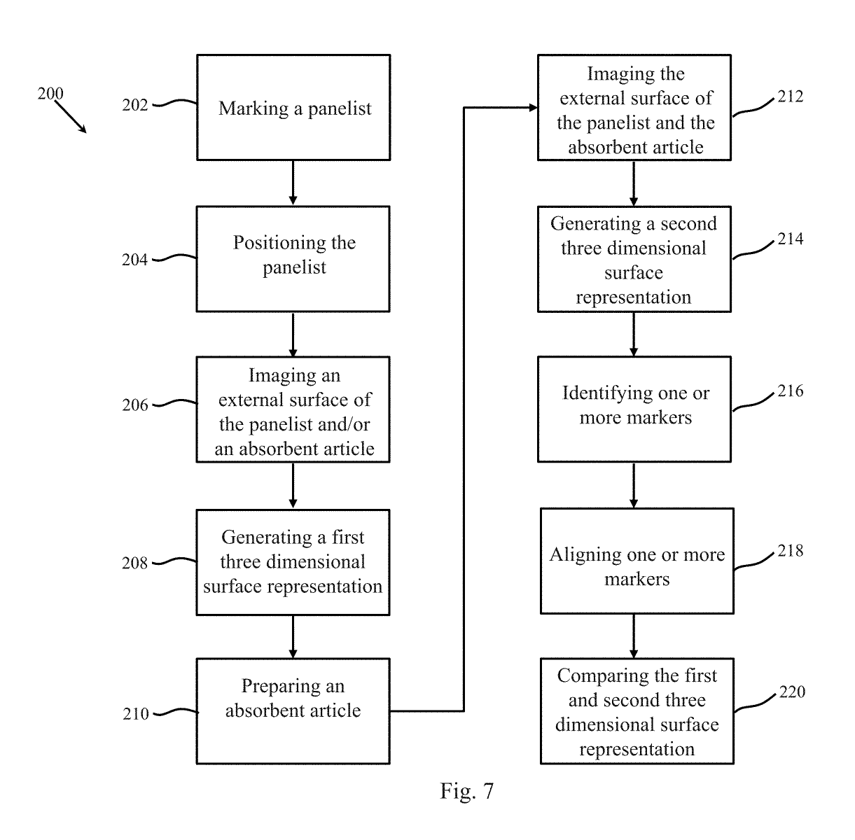

FIG. 7 outlines a method of analyzing the fit and appearance of an absorbent article 200. The method includes marking a panelist 202 using a marking protocol. The marking protocol includes placement of three or more markers on an external surface of the panelist, which will be discussed herein. The method 200 also includes positioning the panelist 204 in an orientation and imaging the external surface of the panelist 206 using visible light and/or infrared light. From the images generated during the imaging session, a first three dimensional surface representation may be generated 208. It is to be appreciated that the panelist may or may not be wearing an absorbent article in the first imaging session. Thus, the panelist may be naked during the first imaging session. An absorbent article may be prepared 210. Preparing the absorbent article 210 may include, for example, having the panelist wear the absorbent article for a certain period of time, having the panelist do certain activities while wearing the absorbent article, and/or soiling the absorbent article with fluid, semi-solid, and/or solid matter. It is to be appreciated that the fluid, semi-solid, and solid may be synthetically made and, subsequently, disposed on the absorbent article. The prepared absorbent article may be placed on the panelist. The method includes imaging the external surface of the panelist wearing the absorbent article 212 and generating a second three dimensional surface representation 214 of the panelist wearing the absorbent article. Each of the first three dimensional surface representation and the second three dimensional surface representation includes the three or more markers that were placed on the panelist and/or the absorbent article. At least three of the three or more markers may be identified and selected 216 in the first three dimensional surface representation and at least three of the three or more markers may be identified and selected in the second three dimensional surface representation. The markers identified in the first three dimensional surface representation correspond to the markers identified in the second three dimensional surface representation. The markers identified in the first three dimensional surface representation may be aligned 218 with the markers identified in the second three dimensional surface representation. The aligning 218 of the first three dimensional surface representation and the second three dimensional representation forms an overlapping three dimensional surface representation. The first three dimensional surface representation may then be compared 220 with the second three dimensional surface representation. Comparing the first and second three dimensional surface representations may include, for example, obtaining the difference in volume and/or surface area.

It is to be appreciated that during any one of the imaging sessions, the panelist may be naked or wearing the consumer product, such as an absorbent article. Further, the absorbent article may be prepared prior to imaging or the absorbent article may be placed on panelist without any prior planned preparation and the panelist wearing the absorbent article may be imaged.

As previously discussed, the method 200 includes marking the panelist 202 with a marking protocol. The marking protocol includes selecting one or more markers to join, either directly or indirectly, to the external surface of the panelist and/or the absorbent article. The one or more markers may be placed within the imaging area on the external surface of the panelist and/or the absorbent article. For example, the one or more markers may be placed on any external surface of the panelist including, for example, the back of the panelist, the front of the panelist, and the sides of the panelist. The one or more markers may be selected such that each marker sufficiently contrasts with the panelist and/or the absorbent article such that the marker is visible in multiple three dimensional surface representations of the panelist and/or the absorbent article. Further, the marker may be selected such that the marker is able to withstand the movement of the panelist and any materials that may be added to the exterior surface of the panelist such as saline, lotion, and/or oil. The marker should be visible and identifiable in the three dimensional surface representation generated by the one or more images and the texture map. For example, the markers may be shiny or matte, include a solid color or a number of colors, and/or include a texture, such as a smooth, homogeneous outer surface. The markers may be lightweight and have good surface contact with the panelist and/or the absorbent article so that the markers do not move relative to the surface on which they are placed.

The markers may include a three dimensional object or a two dimensional mark. For example, a marker including a three dimensional object may be a sphere, a cone, a pyramid, a cylinder, or a prism, such as a rectangular prism, a triangular prism, or hexagonal prism, or any other three dimensional shape that may be identifiable in a three dimensional surface representation and durable enough to remain joined to the panelist. A three dimensional marker may also include a substantially planar object having a surface configured to be joined to the panelist. Each marker may be chemically and/or mechanically joined to the panelist. For example, each marker may include adhesive, such as glue, or additional adhesive may be applied to the marker such that the marker maintains its position on the external surface of the panelist and/or the absorbent article throughout multiple imaging sessions. The marker may also be joined to the panelist mechanically. For example, hook and loop type systems or tape may be used to join the marker to the panelist. As used herein, the term “joined” encompasses configurations whereby an element is directly joined to the other element by affixing the element directly to the other element, and configurations whereby the element is indirectly secured to the other element by affixing the element to intermediate element(s) which in turn are affixed to the other element.

The marker may be any size such that the marker is visible in the three dimensional surface representation and minimally interferes with imaging the panelist, preparation of the absorbent article, and/or movement of the panelist. For example, the marker may be placed so as not to obstruct an area that is to be later measured. The markers may be made with any number of materials such as Styrofoam, plastic, either hollow or solid, foamed materials such as urethane foams, and/or wood. Further, the external surface of the marker may be unaffected or coated with an additional material such as paint, ink, dye, varnish, stickers, and/or wax to increase the visibility of the marker and/or to protect the marker during wear by the panelist. For example, metallic paint may be used to coat at least a portion of the marker. The metallic paint creates a reflective surface that aids in maintaining the visibility of the marker while imaging and, subsequently, identifying the marker in the three dimensional surface representation.

It is also to be appreciated that the marker may be a two dimensional mark placed on the panelist and/or the absorbent article. Ink or paint, for example, may be used to form a two dimensional mark on the panelist and/or the absorbent article. Both two dimensional marks and three dimensional objects may be used in the marking protocol. Markers may be removed from the panelist after all imaging session(s) are complete.

Anatomical features may also serve as markers. Anatomical features include, for example, a belly button, the lowest point on the crotch, a hip bone protrusion, urethra, and anus.

The markers may be used for defining one or more planes, locating features of the absorbent article and panelist, monitoring features of the absorbent article or the panelist such as, for example, how the ears stretch and how the waist sags, and aligning the three dimensional surface representations.

The number and type of markers used will depend, at least in part, on the characteristics of the panelist and/or the consumer product to be imaged and the area that is to be imaged. For example, for imaging a single consumer product that does not move and fits within the entire imaging area, a single marker may be used to align the three dimensional surface representations. However, for imaging a panelist that is able to move, additional markers may be needed. For example, at least three markers may be used to align the three dimensional surface representations. Further, for imaging a panelist that is able to move and where portions of the panelist may be outside the imaging area (i.e., the portion of the panelist above the torso region or the portion below the thigh region of the legs), at least three markers may be used to align the three dimensional surface representations. It is also to be appreciated that certain consumer products may require no markers and having a panelist which is a mannequin may also not require the use of markers.

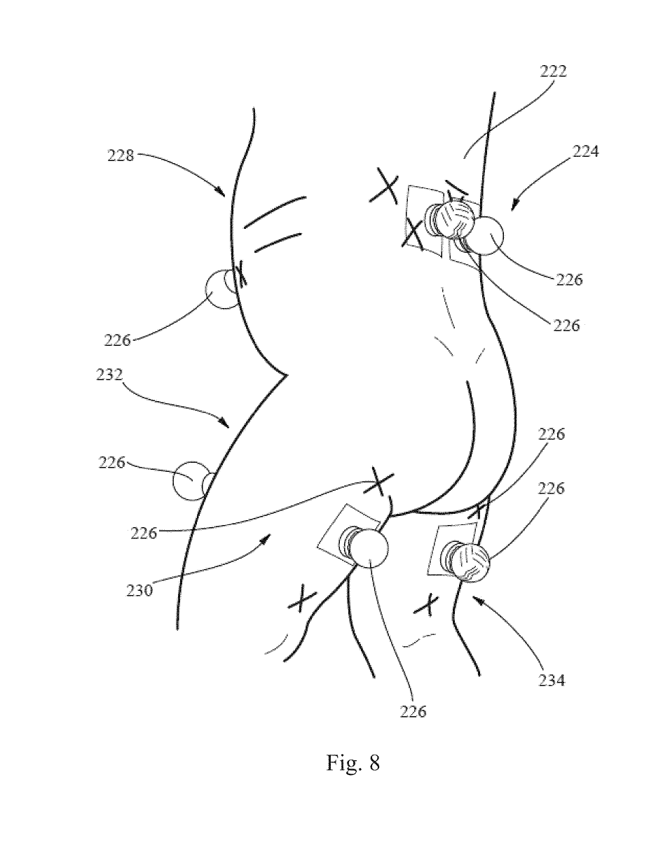

For example, in some embodiments, the marking protocol may include identifying markers that provide sufficient contrast to the external surface of the panelist 222 and/or the external surface of the absorbent article. These markers may be two dimensional marks, three dimensional objects, or a combination of two dimensional marks and three dimensional objects. A first marker set 224 may be placed on the external surface of the panelist 222, as illustrated in FIG. 8. The first marker set 224 may include at least three markers 226. The first marker set may be joined to the external surface of the panelist such that the first marker set may define a first plane. More specifically, the first marker set may include three markers. The three markers may be placed in a first location about the torso 228 of the panelist. The first marker set, which may define a first plane, may be positioned below the neck base girth and above the hip girth.

For example, the first marker set may be disposed on the torso of the panelist and/or absorbent article in the following manner. Starting at the hip girth (as defined in ASTM D5219-02) at least three markers may be placed on the panelist's skin approximately 30% of the vertical distance from the hip girth to the neck base girth (as defined in ASTM D5219-02). These markers may be spaced around the circumference of the torso. Further, starting at the hip girth, at least three markers may be placed on the panelist's skin and/or the absorbent article approximately 50% of the vertical distance from the hip girth to the neck base girth. These markers may be spaced around the entire circumference of the torso. The markers may be placed anywhere between the hip girth and the neck base girth. It is to be appreciated that more than one set of markers may be disposed on the torso of the panelist. For example, where different absorbent articles are used in the imaging sessions, one set of markers may be visible while a first absorbent article is worn by the panelist but may not be visible when a second absorbent article is worn, such as due to a change in fit of the second absorbent article when worn by the panelist.

The marking protocol may include placing a second marker set on the external surface of the panelist and/or the absorbent article. The second marker set may include at least three markers. The second marker set may be joined to the external surface of the panelist such that the second marker set may define a second plane. The second plane may be placed in a second location on the external surface of the panelist. The second location may be a first thigh region of the panelist. The second marker set, which defines the second plane, may be placed between the crotch and the back of the knee or crease in the first thigh region.

For example, the second marker set may be disposed about the first thigh region of the panelist in the following manner. Starting at the midpoint of the back of the knee, also referred to as the crease (as defined in ASTM D5129-02) place a set of at least three markers on the panelist's skin approximately 20% of the vertical distance from the midpoint of the back of the knee (or crease) to the thigh girth (as defined in ASTM D5219-02). These markers may be spaced around the circumference of the first thigh region. At least three markers may also be placed 40% of the vertical distance from the midpoint of the back of the knee (or crease) to the thigh girth. These markers may be spaced around the circumference of the thigh region.

The marking protocol may include placing a third marker set on the external surface of the panelist and/or the absorbent article. The third marker set may include at least three markers. The third marker set may be joined to the external surface of the panelist such that the third marker set may define a third plane. The third marker set, which defines the third plane, may be placed in a third location on the external surface of the panelist. The third location may be a second thigh region 234 of the panelist 222. The third plane may be placed between the crotch and the back of the knee or crease in the second thigh region 234.

For example, the third marker set may be disposed about the second thigh region of the panelist in the following manner. Starting at the midpoint of the back of the knee, also referred to as the crease (as defined in ASTM D5219-02) place a set of at least three markers on the panelist's skin approximately 20% of the vertical distance from the midpoint of the back of the knee (or crease) to the thigh girth (as defined in ASTM D5219-02). These markers may be spaced around the circumference of the second thigh region. At least three markers may also be placed 40% of the vertical distance from the midpoint of the back of the knee (or crease) to the thigh girth. These markers may be spaced around the circumference of the second thigh region.

It is to be appreciated that each of the planes may be substantially horizontal or at an angle. It is also to be appreciated that the markers used to align the three dimensional surface representations may or may not be the same markers that are used to establish the planes. Markers may be placed anywhere on the panelist and/or the absorbent article to aid in alignment of the three dimensional surface representations and/or for defining any number of planes that may be used for comparing the three dimensional surface representations and taking measurements.

Further, in some embodiments, the marking protocol may include placing a fourth mark on a longitudinal centerline of the external surface of the panelist. The fourth mark may be placed along the longitudinal centerline of either the back or the front of the panelist. The fourth mark may be positioned such that the fourth mark is above the absorbent article and present in the imaging area. In some embodiments, for example, the fourth mark may be placed along the center back waist length (as defined in ASTM D5219-02). The fourth mark may be disposed on the external surface of the panelist at approximately 30% of the vertical distance from the hip girth to the neck base girth and along the longitudinal centerline of the panelist.

The markers may also be placed adjacent the panelist such as on a rigid fixture placed within the area that is captured by the image capture device. Further, markers placed adjacent the panelist may be used in addition to or as an alternative to markers disposed on the panelist and/or the absorbent article. The markers placed adjacent the panelist may be used in the three dimensional surface representation for alignment, cutting, and/or measurement. These markers placed adjacent the panelist may also be removed from the three dimensional surface representation once they have served their purpose, such as for alignment of two or more three dimensional surface representations.

Once the panelist and/or the absorbent article are marked with the markers using the marking protocol, the panelist may be positioned 204 in an orientation. The orientation may be selected such that each of the markers is visible to at least one of the image capture devices in at least one of the imaging sessions, which may use a visible light and/or infrared light to capture data. Further the orientation should be one that is substantially reproducible. Thus, floor indicators may be used to place the panelist's feet and a bar or other handle device may be used for placement of the panelist's hands. Further, the panelist may be positioned in a location that is suitable for imaging the panelist. It is to be appreciated that the orientation may be a standing position, a sitting position, a bending position, or any other position likely to provide information indicative of performance characteristics of the absorbent article. The orientation may be one that minimizes the occlusion of relevant features. Stated another way, the orientation may be one in which relevant features are visible to the image capture devices.

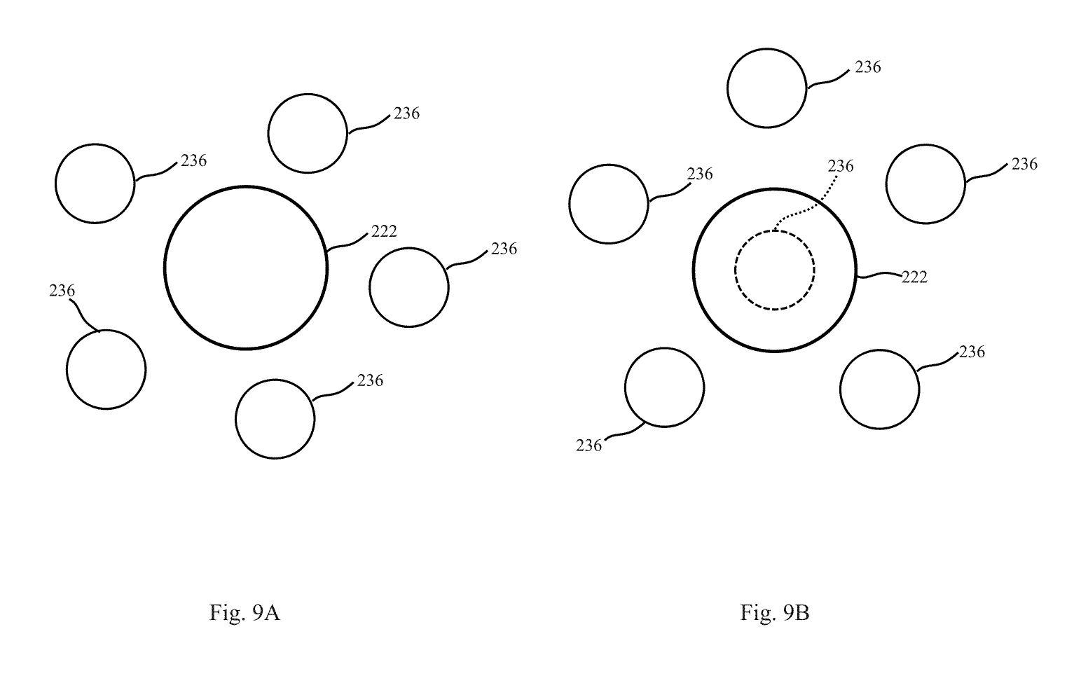

The external surface of the panelist may be imaged 206 using visible light and/or infrared light. The panelist may be a naked panelist when imaged or the panelist may be wearing an absorbent article, such as a diaper, when imaged. One or more image capture devices 236, which may use visible light and/or infrared light, may be positioned about the panelist, when the panelist is positioned in the orientation. An image capture device may also include a laser based scanning device. Any number of image capture devices may be used such that the surface of the panelist is adequately imaged. For example, greater than two and/or greater than three and/or greater than four and/or greater than five image capture devices may be used. In some embodiments, for example, five image capture devices, which may use visible light and/or infrared light, may be placed about the panelist, as illustrated in FIG. 9A, which is a schematic, top view illustration. In some other embodiments, for example, additional image capture devices 236 may be used to image the panelist. As illustrated in FIG. 9B, six image capture devices 236, which may use visible light and/or infrared light, may be positioned about the panelist 222. As illustrated, one of the image capture devices 236 may be positioned below the panelist 222 to obtain data on the crotch of the panelist, which may not be entirely visible to the other image capture devices disposed about the panelist. It is to be appreciated that more or less image capture devices may be used to obtain images of the panelist. For example, a single image capture device may use mirrors to generate stereo images, which includes two images at a known angle and distance. Further, the image capture device(s) may also be moveable with respect to the panelist. Additionally, the image capture device(s) may be moved while the panelist is being imaged.

The image capture device, using visible light and/or infrared light, captures a photographic surface image of the external surface of the panelist, who may be naked or wearing an absorbent article. The image capture device may be a device that projects light and captures an image during the projection of light. An example of an image capture device is a digital camera. For example, the digital camera may use visible light to produce colored or monochrome images. The digital camera may use infrared light to produce monochrome images. These images may be used to create a texture map, which may be generated in part using the colored images, and three dimensional point cloud that represents the panelist, which may be generated in part using the monochrome images. The digital images captured by the digital cameras may be communicated to a computer system, which may also be referred to herein as a computer imaging system, which uses software to generate and obtain 208 a three dimensional surface representation 238 of the panelist 222. The image capture devices and/or the software may be provided by, for example, 3dMD LLC, 3200 Cobb Galleria Parkway # 203, Atlanta, Ga. 30339 USA; Vectra H1 Handheld imaging system and Vectra Software from Canfield Imaging Systems, 253 Passaic Ave. Fairfield, N.J. 07004; Artec Eva handheld scanner and Artec studio 10 software from Artec 3d, 335 Bryant Street, Palo Alto, Calif. 94301; Kinect2 and iPisoft from Microsoft, One Microsoft Way, Redmond, Wash. 98052; and Ipisoft Mocap Studio software from iPi Soft LLC, Moscow, 127434, Russia.

For example, in some embodiments, each of the five image capture devices disposed about the panelist may include two monochrome digital cameras, one color digital camera, and a speckle projector (flash unit). The monochrome images are used to build a 3D point cloud, which is then meshed. It is to be appreciated that a mesh is a type of three dimensional surface representation. Each point on the projected speckle pattern may be visible by at least three of the digital cameras in order to triangulate the position of each point on the surface (using the speckle projection and photogrammetry). Then the color camera is fired. The image from the color camera is mapped to the mesh. Because the image is higher resolution than the mesh, the mapped pixel positions inside each mesh triangle are interpolated, thus providing sub-triangle resolution and making it possible to have accurate color information.

In some other embodiments, a Vectra H1 3d camera uses stereo photogrammetry to calculate the 3D coordinates of points on the surface of a panelist using two digital images taken from a precisely known position and angle to the panelist. The data gathered by using the camera may be used to create a three dimensional surface representation.

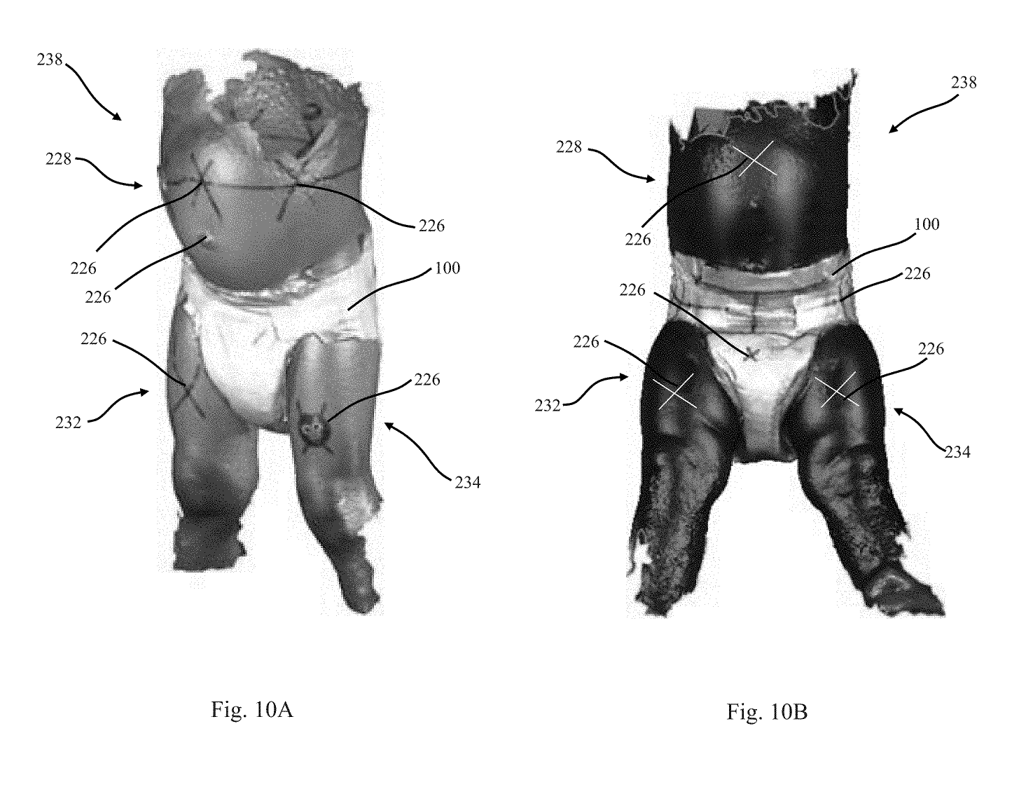

The three dimensional surface representation 238, generated by the software and displayed using the computer system, may be of any portion of the panelist 222 captured by the image capture devices 236. For example, the three dimensional surface representation, as illustrated in FIGS. 10A and 10B, may include the portion of the panelist wearing the absorbent article and/or a portion of the panelist above one or more markers disposed in the torso region and/or a portion of the panelist below one or more markers disposed in the thigh region. However, it is to be appreciated that the entire body or any other portion of the panelist may be imaged and displayed as a three dimensional surface representation. The panelist may be imaged any number of times. Each time the panelist is imaged and a three dimensional surface representation may be generated from those images, this may be referred to herein as an imaging session. At least two imaging sessions and at least two three dimensional surface representations may be generated from the gathered imaging data.

As previously discussed, the panelist may be imaged while naked or while wearing an absorbent article. In some embodiments, the panelist may be imaged while naked to obtain a first three dimensional surface representation and the panelist wearing the absorbent article may be imaged to obtain a second three dimensional surface representation. Prior to imaging the panelist wearing the absorbent article, the absorbent article may be prepared 210. The absorbent article may be prepared in a number of ways depending on the performance characteristic of the absorbent article that is desired to be evaluated. Preparation of the absorbent article may include, but is not limited to: loading the absorbent article one or more times in a certain location and with a certain amount of fluid and/or semi-solid and/or solid; loading the absorbent article and waiting a certain period of time before placing the loaded absorbent article on the panelist; having the panelist wear the absorbent article for a certain period of time; having the panelist wear the absorbent article for a certain period of time and perform certain tasks; having the absorbent article applied to the panelist in a certain manner such as, for example, applying the fasteners consistently such that spacing between fasteners is substantially the same and applying such that the portion between the panelist's legs exhibits a certain fold pattern, which may include, for example, a v-shaped pattern; loading the absorbent article one or more times with a certain amount of semi-solid material in a certain location; removing the absorbent article and placing the same absorbent article back on the panelist any number of times; and loading the same absorbent article one or more times between each of the multiple imaging sessions. It is to be appreciated that the aforementioned list is not intended to be a complete list. Preparation of the absorbent article may include one or more activities that may result in a change in the characteristics, such as fit or appearance, of the absorbent article.

It is to be appreciated that that panelist and/or the panelist wearing an absorbent article may be imaged any number of times. However, to obtain and compare the performance characteristics of an absorbent article, at least two imaging sessions should be completed such that two three dimensional surface representations may be generated. For example, a first imaging session may include the naked panelist and a second imaging session may include the panelist wearing an absorbent article. The first imaging session may include a panelist wearing a first absorbent article and the second imaging session may include a panelist wearing a second absorbent article. The first absorbent article may be the same brand or a different brand than the second absorbent article. Further, the first absorbent article and the second absorbent article may undergo the same preparation activities or different preparation activities. Further still, a first imaging session may include a panelist wearing an absorbent article and a second imaging session may include a panelist wearing the same absorbent article, but the absorbent article has undergone some preparation prior to the second imaging session. Additional imaging sessions, in addition to the two, may also be completed. For example, a first imaging session may include a naked panelist, a second imaging session may include a panelist wearing a first absorbent article, and a third imaging session may include a panelist wearing a second absorbent article. The first absorbent article and the second absorbent article may be the same brand or different brands. Further, the first absorbent article and the second absorbent article may undergo different preparation activities prior to the images being obtained.

Each imaging session generates a number of two dimensional images which may be passed, such as by a processor, to software, as previously mentioned, that takes the two dimensional images and generates a three dimensional surface representation. For example, images of the naked panelist generates a first three dimensional surface representation of the naked panelist. The three dimensional surface representation may only be as complete as the photographic surface images gathered using the one or more image capture device(s). Thus, areas of the panelist that were not visible by the image capture devices may appear as void areas in the three dimensional surface representation. The void areas may be repaired in the three dimensional surface representation so that the surface representations may be compared. Software may be used to repair the three dimensional surface representation such that the void areas are minimized or eliminated. More specifically, the three dimensional surface representation may be generated and, subsequently opened using a computer system, also referred to herein as a computer modeling system, which uses software that may repair the defects of the three dimensional surface representation. Software that repairs the three dimensional surface representation is available from, for example, Meshlab; Geomagic Studio Software from 3d systems, Rock Hill, S.C. 29730; and Rhinoceros Software from McNeel North America, Seattle, Wash. 98103.

Other defects present in the three dimensional surface representation may also be repaired. For example, the software may identify additional defects such as duplicate triangles, orphan triangles, non-manifold edges, self-intersections, highly creased edges, spikes, small components, small tunnels, small holes, etc. It is to be appreciated that each software package may identify different defects and repair these defects in a different manner. Each three dimensional surface representation may be repaired prior to or after two or more three dimensional surface representations are aligned.

The markers may be present on the external surface of the panelist and/or absorbent article during each imaging session and may be visible in each three dimensional surface representation. At least three of the markers may be identified 216 in the first three dimensional surface representation. Similarly, at least three of the markers may be identified 216 in the second three dimensional surface representation. The markers identified in the first three dimensional surface representation may be the same as the markers identified in the second three dimensional surface representation. The markers identified may be collinear or non-collinear. However, in some embodiments, it may be easier to align the three dimensional surface representations using markers that are non-collinear. It is to be appreciated that more than or less than three markers may be identified in each three dimensional surface representation.

The identified or selected markers may be used to align the first three dimensional surface representation with the second three dimensional surface representation. More specifically, a first marker present in the first three dimensional surface representation may be aligned with a corresponding first marker present in the second three dimensional surface representation. Similarly, a second marker present in the first three dimensional surface representation may be aligned with a corresponding second marker present in the second three dimensional surface representation. More markers may be aligned if necessary. Alignment of the first three dimensional surface representation and the second three dimensional surface representation may be an iterative process. Further, certain markers may be better suited for alignment than others. For example, given that the belly of a panelist expands and contracts while the panelist is breathing, markers present in the expanding and contracting area may not be the most ideal for alignment due to the change in position of the marker. Aligning may include translation (i.e., movement of the surface representation in one or more directions), rotation, and/or transformation (i.e., assignment of new coordinate system) of the surface representations to obtain an overlapping three dimensional surface representation. The order of alignment of the selected or identified markers may also aid in obtaining a useable overlapping three dimensional surface representation. The overlapping three dimensional surface representation includes each of the first three dimensional surface representation and the second three dimensional surface representation. The computer system or computer modeling system including the previously disclosed software may be used to aid in alignment of two or more three dimensional surface representations.

As previously discussed, the panelist may be positioned in an orientation during the first imaging session. The panelist may be returned to substantially the same orientation during each subsequent imaging session. Orienting the panelist in substantially the same way during each of the imaging session aids in the alignment of the three dimensional surface representations. Generally, the more similar the orientation of the panelist across imaging sessions, the more precise the alignment and measurements of the three dimensional surface representations.

It is to be appreciated that the panelist may be a human individual and thus, it may be difficult to orient the human individual in the same orientation. To account for some movement from imaging session to imaging session, the three dimensional surface representation generated by the images may be modified. For example, the first three dimensional surface representation may be a naked panelist and the second three dimensional surface representation may be a panelist wearing an absorbent article. The legs of the panelist illustrated in the first three dimensional surface representation may be separated by a greater distance than the legs of the panelist illustrated in the second three dimensional surface representation. Either one of or both of the legs illustrated in the first three dimensional surface representation and the second three dimensional surface representation may be adjusted using the software such that the distance separating the legs illustrated in the first three dimensional surface representation are substantially the same as the distance separating the legs illustrated in the second three dimensional surface representation. Thus, either or both of the first three dimensional surface representations and the second three dimensional surface representation may be distorted such that each surface representation is positioned in substantially the same orientation. It is to be appreciated that the three dimensional surface representation may be repositioned, such as by rotating and/or translating, a portion of the surface representation. For example, a first portion of the three dimensional surface representation may remain fixed or stationary while a second portion of the three dimensional surface representation is repositioned.

The first three dimensional surface representation may then be compared 220 with the second three dimensional surface representation. The first three dimensional surface representation may be compared with the second three dimensional surface representation or any other generated, aligned three dimensional surface representation to obtain a number of measurements including, for example, volume, surface area, length, angle, perimeter or a portion thereof, and location of one or more features.

For certain comparisons of the first three dimensional surface representation and the second three dimensional surface representation, the first three dimensional surface representation, the second three dimensional surface representation, or the overlapping three dimensional surface representation may be made watertight. Watertight refers to the representation being a closed three dimensional representation that encloses a volume. A watertight three dimensional surface representation is hollow. However, the watertight three dimensional surface representation may be filled such that the watertight three dimensional surface representation is no longer hollow. A watertight three dimensional surface representation that is not hollow may be referred to herein as a filled three dimensional surface representation. It is to be appreciated that when the three dimensional surface representation is filled, a computer system may be used to create an enclosed volume that surrounds the three dimensional surface representation. Within the enclosed volume a value greater than zero is assigned to those portions within the three dimensional surface representation and a zero value is assigned to those portions outside the three dimensional surface representation but within the enclosed volume. The watertight three dimensional surface representation and the filled three dimensional surface representation may each be used to obtain the volume or difference in volume between multiple three dimensional surface representations.

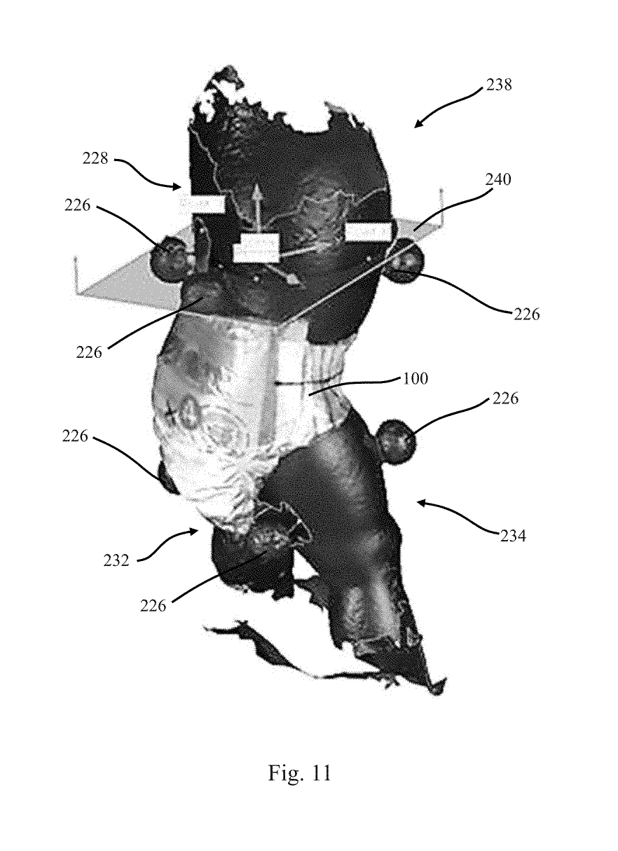

More specifically, in some embodiments, the first three dimensional surface representation and the second three dimensional surface representation may be made watertight. To make the representation watertight, two or more cutting planes may be identified, trimmed along, and sealed. More specifically, for example, at least three of the markers present in the first three dimensional surface representation may be identified and a first cutting plane may extend with reference to the markers. For example, where the marker comprises a two dimensional mark in in the shape of an “x”, the cutting plane may extend through the center of the “x”. Where the marker comprises a three dimensional sphere, a point on the sphere may be identified such that the cutting plane extends through the same point on the sphere in both the first and second three dimensional surface representations. As illustrated in FIG. 11, three markers 226 positioned about the torso region 228 may be identified and a first cutting plane 240 may extend through the torso region 228 based on the location of the three markers 226. The three dimensional surface representation may be trimmed along the cutting plane 240 and the portion of the three dimensional surface representation either above or below the cutting plane may be removed. It is to be appreciated that the markers identified for cutting, trimming, and sealing the three dimensional surface representation may be the same or different than the markers identified for use in aligning the three dimensional surface representation.

Similarly, one or more additional cutting planes may be identified using the markers disposed on the panelist and/or the absorbent article. For example, a second cutting plane may extend with reference to the markers positioned in the first thigh region. The three dimensional surface representation may be trimmed along the second cutting plane. For example, the second cutting plane may extend through the first thigh region of the panelist. Thus, the portion of the three dimensional surface representation illustrating the portion of the first leg of the panelist below the second cutting plane may be removed. However, the second leg of the panelist or any other portion of the three dimensional surface representation, even though it may be below the second cutting plane, may not be removed. A cutting plane may be specific to a certain portion of the three dimensional surface representation. Further, a third cutting plane may extend with reference to the markers positioned in the second thigh region. The three dimensional surface representation may be trimmed along the third cutting plane. Similarly, for example, the third cutting plane may extend through the second thigh region of the panelist, and, thus, the portion of the three dimensional surface representation illustrating the portion of the second leg of the panelist below the third cutting plane may be removed. However, any other portion, such as a portion of an absorbent article, of the three dimensional surface representation, even though it may be below the third cutting plane, may not be removed.

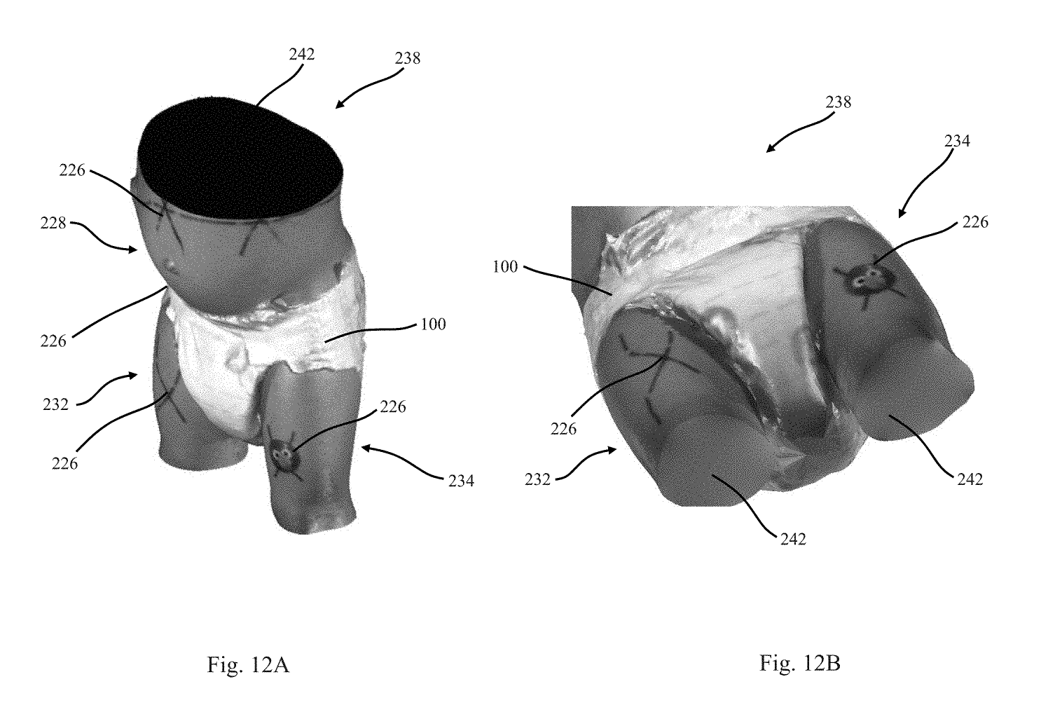

It is to be appreciated that, in some embodiments, a single cutting plane may be used to extend through both of the first thigh region and the second thigh region. The three dimensional surface representation may be sealed along each of the trimmed cutting planes to form a watertight three dimensional surface representation, such as illustrated in FIGS. 12A and 12B. More specifically, each of the three dimensional surface representations may be sealed forming one or more sealed planes 242. Each of the three dimensional surface representations may undergo the aforementioned steps to become watertight. Thus, for example, at least three of the markers may be selected in each of the three dimensional surface representations, cutting planes may be identified based on the selected markers in each of the three dimensional surface representations, and trimmed along each of the cutting planes to form watertight three dimensional surface representations.

In some embodiments, the overlapping three dimensional surface representation may be used to render each of the first three dimensional surface representation and the second three dimensional surface representation watertight. To make the overlapping three dimensional representations watertight, two or more cutting planes may be identified, trimmed along, and sealed. More specifically, for example, at least three of the markers present on any one of the aligned three dimensional surface representations may be used to form a cutting plane. For example, at least three of the markers present on the first three dimensional representation and/or at least three of the markers present on the second three dimensional surface representation may be used to form a cutting plane. The cutting plane formed by at least three of the markers may extend through both of the first three dimensional surface representation and the second three dimensional surface representation. Both of the first three dimensional surface representation and the second three dimensional surface representation may be trimmed using the single cutting plane such that the portion of the three dimensional surface representations either below or above the cutting plane is removed. Similar to the above, a second cutting plane may extend through each of the first thigh region and the second thigh region, or a second cutting plane and a third cutting plane may extend through the first thigh region and the second thigh region, respectively. However, it is to be appreciated that the second cutting plane and/or the third cutting plane may be used to trim both of the first three dimensional surface representation and the second three dimensional surface representation. The three dimensional surface representation may be sealed along each of the trimmed cutting planes to form a watertight overlapping three dimensional surface representation.

Subsequent to the three dimensional surface representation(s) being made watertight, the three dimensional surface representation may be filled such that the watertight three dimensional surface representation is no longer hollow and creates a filled three dimensional surface representation. Filling a three dimensional surface representation transforms the hollow three dimensional surface representation into a solid object made up of voxels. It is to be appreciated that it not necessary to fill the watertight three dimensional surface representation(s) to obtain volume information.

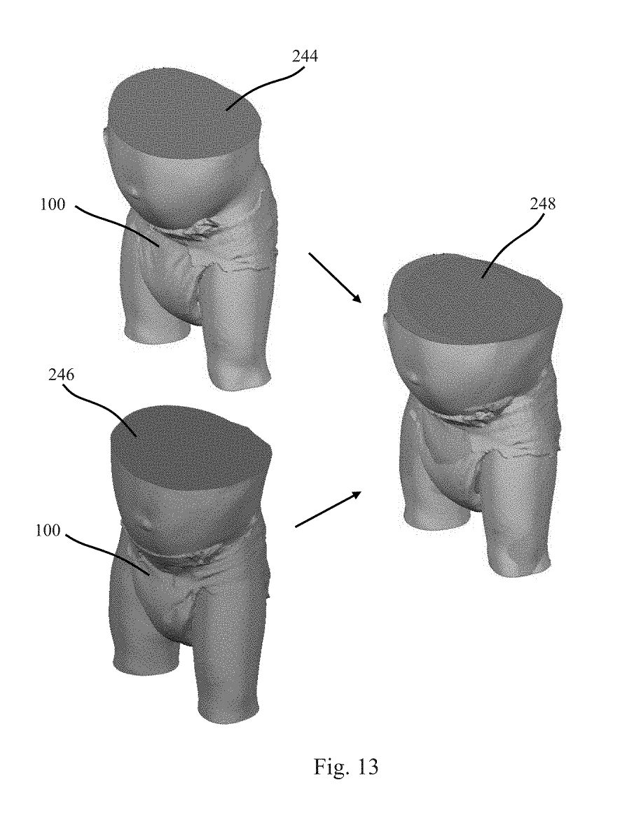

As previously discussed, the first three dimensional surface representation 244 may be aligned with the second three dimensional surface representation 246, as illustrated in FIG. 13. It is to be appreciated that each three dimensional surface representation may be repaired prior to or after undergoing alignment. Similarly, each three dimensional surface representation may be trimmed, sealed, and/or filled prior to or after undergoing alignment.

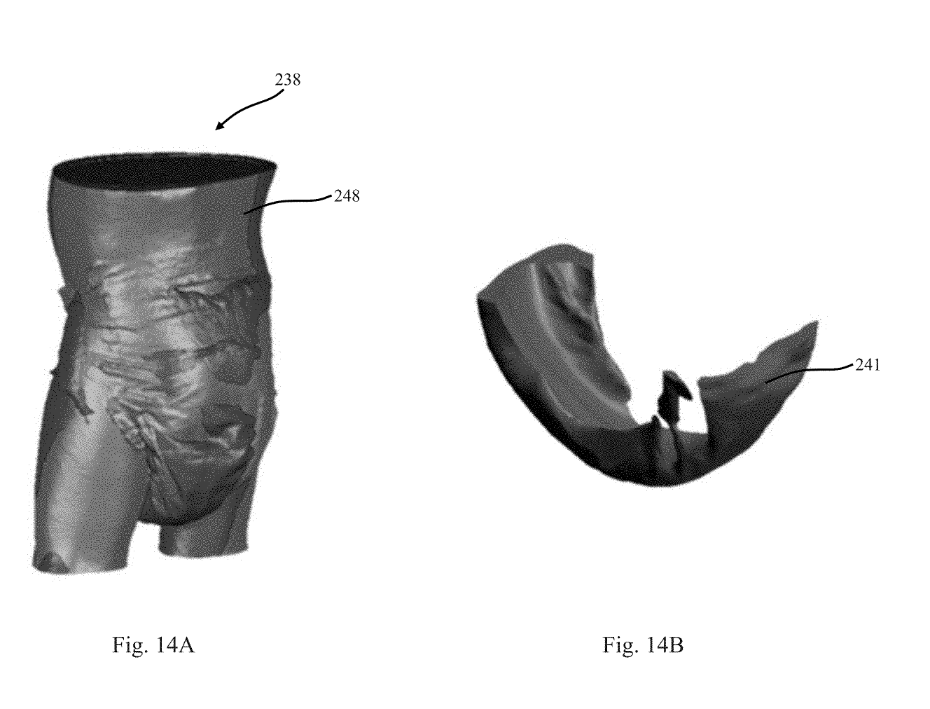

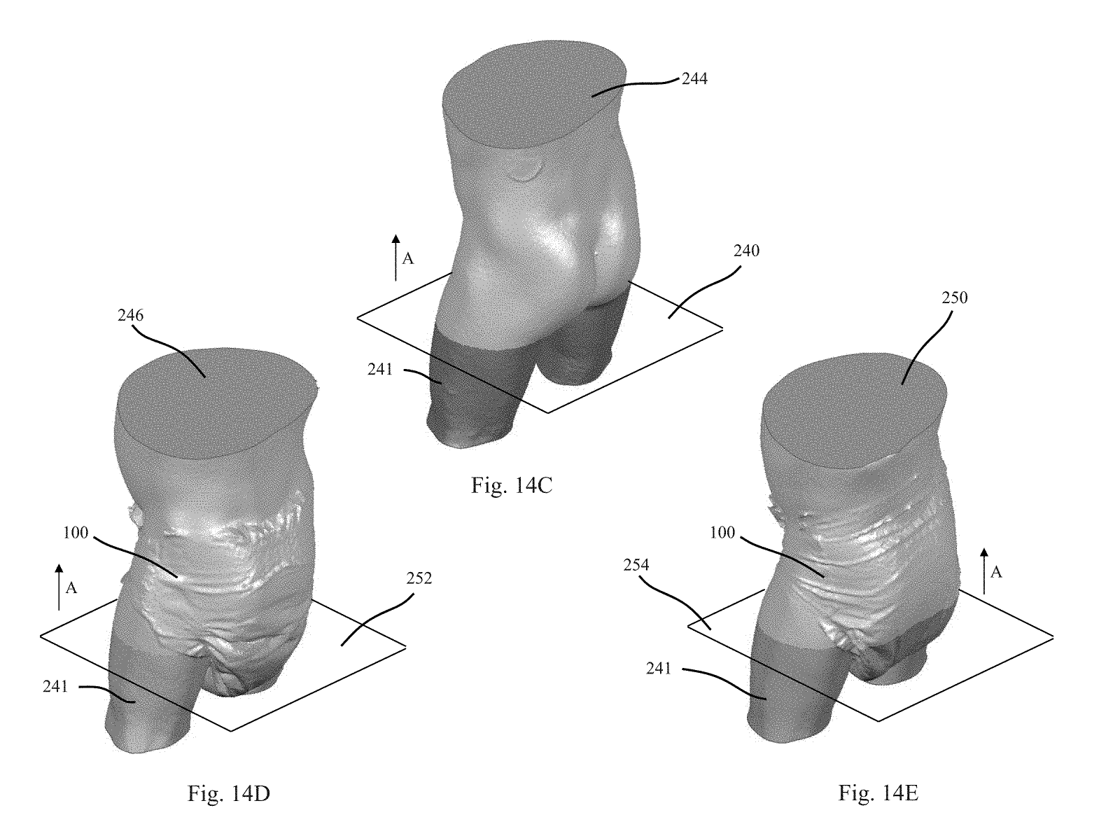

Comparing the first three dimensional surface representation and the second three dimensional surface representation may include obtaining the difference in volume between the first three dimensional surface representation and the second three dimensional surface representation. The volume may be obtained by obtaining a first volume of the first three dimensional surface representation and, separately, obtaining a second volume of the second three dimensional surface representation and, subsequently, subtracting the quantity of the first volume from the quantity of the second volume. For example, because the absorbent article may change between the two surface representations but the panelist remains the same, a difference in the volume of the absorbent article may be obtained, which may include the gap or space between the panelist and the absorbent article. The volume may also be obtained by directly subtracting the first three dimensional surface representation from the second three dimensional surface representation in an overlapping configuration. For example, in the overlapping three dimensional surface representation, which is the overlapping of the first three dimensional surface representation and the second three dimensional surface representation, the overlapping volume may be illustrated, as shown in FIG. 14A. Further, the overlapping volume may be determined, such as by subtracting the volumes,and the remaining volume may be the difference in volume between the first and second surface representations, as illustrated in FIG. 14B.

It is to be appreciated that watertight three dimensional surface representations or filled three dimensional surface representations may be used to obtain the volume or difference in volume. In embodiments where watertight three dimensional surface representations are used to obtain the volume by directly subtracting the first and second three dimensional surface representations, the remaining portions of the overlapping three dimensional surface representation, or the portions of the overlapping three dimensional surface representation that are not exactly aligned may be cleaned up such that the irrelevant portions are eliminated and the relevant portions are made watertight and the volume obtained. In embodiments where filled three dimensional surface representations are used to obtain the volume by directly subtracting the first and second three dimensional surface representations, the remaining portions of the overlapping three dimensional surface representation, or the portions of the overlapping three dimensional surface representation that are not exactly aligned may be modified such that the irrelevant portions are eliminated and the volume of the relevant portions are obtained. If the relevant portions were previously made watertight and filled this does not need to be done again. The voxel information of the relevant portions may be used to obtain the volume.

Referring to FIGS. 14C-14E, for example, the volume of a region of interest may be obtained by referencing features that are visible in one three dimensional surface representation and hidden in another three dimensional surface representations. As illustrated in FIG. 14C, a first three dimensional surface representation 244 may include a naked panelist. The representation of the naked panelist allows the crotch to be located on the first three dimensional surface representation.

However, as illustrated in FIGS. 14D and 14E, a second three dimensional surface representation 246 may include the panelist wearing a first absorbent article and a third three dimensional surface representation 250 may include the panelist wearing a second absorbent article or the first absorbent article that has undergone certain preparation activities, as previously discussed. The crotch of the panelist is not visible in the second three dimensional surface representation and the third three dimensional surface representation due to the absorbent article. To locate the crotch of the panelist, a first cutting plane 240 may extend adjacent the crotch of the panelist as represented in the first three dimensional surface representation 244. The first cutting plane may also extend through the second three dimensional surface representation and the third three dimensional surface representation when each of the three dimensional surface representations are aligned. The first, second, and third three dimensional surface representations may be trimmed and sealed along the first cutting plane 240. The volume of each of the first, second, and third three dimensional surface representations may then be obtained and compared.

Still referring to FIGS. 14C-14E, in some embodiments, the volume of each of the first, second, and third three dimensional surface representations may be obtained independently and compared. Thus, the location of the first cutting plane 240 may be obtained using features of the first three dimensional surface representation, such as one or more markers, which may include an anatomical feature and/or a sealed plane. Using the location of the first cutting plane 240, a second cutting plane 252 may be positioned in the location corresponding to the crotch in the second three dimensional surface representation 246. Similarly, using the location of the first cutting plane 240, a third cutting plane 254 may be positioned in the location corresponding to the crotch in the third three dimensional surface representation 250. Each three dimensional surface representation may be trimmed, such that the portion above the cutting planes, indicated by directional arrow A, is removed, and sealed along their respective cutting planes to obtain the volume of the portion 241 of each of the first three dimensional surface representation, the second three dimensional surface representation, and the third three dimensional surface representation. Each of the volumes obtained may then be compared to analyze the change in volume between each of the surface representations.

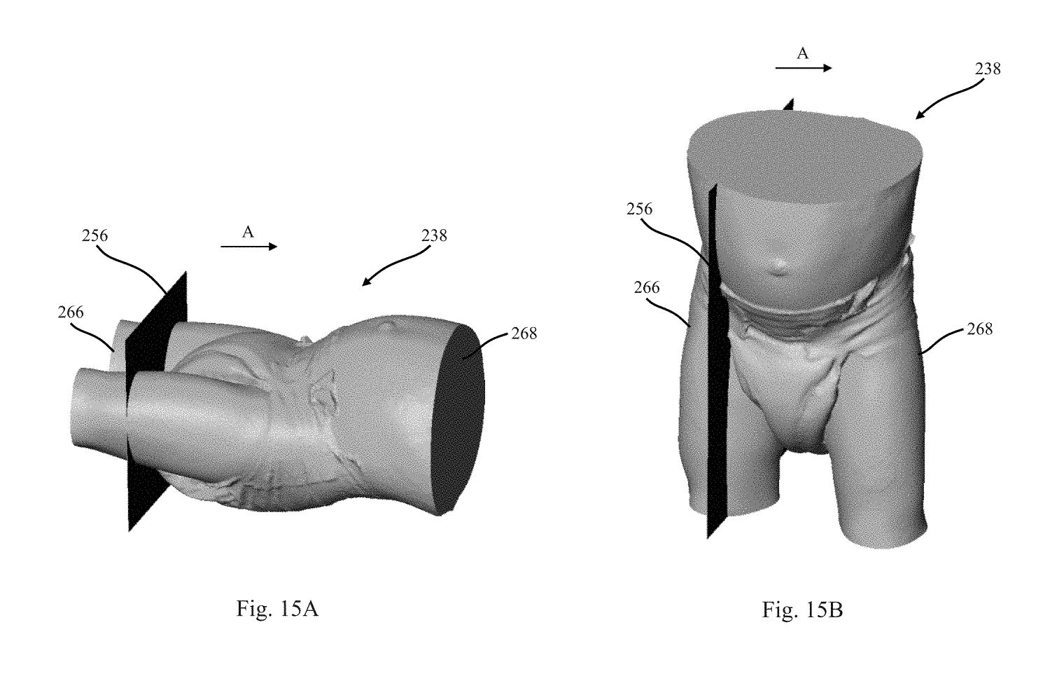

The three dimensional surface representations may also be used to generate graphical representations of the change in volume or area over the three dimensional surface representation. Referring to FIGS. 15A and 15B, each of the three dimensional surface representation may be divided into one or more portions. More specifically, a slice may advance through the three dimensional surface representation. The slice may have some thickness such that the volume at the location of the slice may be obtained. The slice may be advanced such that the end of the first slice is the beginning of the next slice. For example, as illustrated in FIGS. 15A and 15B, the slice may advance from a first end portion 266 to a second end portion 268 in a direction indicated by arrow A. The volume as the slice advances from the first end portion to the opposite, second end portion of the three dimensional surface representation may be plotted. The volume may be plotted as a function of position based on a reference point in the three dimensional surface representation or as a function of the number of the slice. It is to be appreciated that the slice may be so thin that the area may be plotted as a function of position or the number of the slice. Each three dimensional surface representation may undergo dividing and the results of each may be plotted in a single graph to illustrate the change in volume or area of the different three dimensional surface representations. It is to be appreciated that the three dimensional surface representation may be divided into any number of slices. For example, the three dimensional surface representation may be divided by a single slice such that the three dimensional surface representation is divided into a first and second portion. The three dimensional surface representation may be divided by three or more slices in some other embodiments. The volume between each slice may be plotted as previously described.

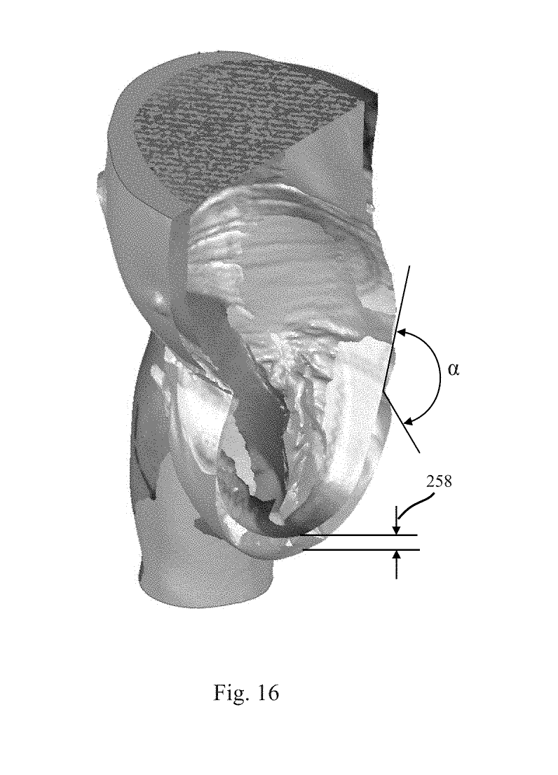

The first three dimensional surface representation and the second three dimensional surface representation may be trimmed while aligned using additional cut planes, such as illustrated in FIG. 16. For example, a central cut plane extending substantially in the vertical direction may be applied to the overlapping surface representations. The central cut plane may extend through both the first three dimensional surface representation and the second three dimensional surface representation. The overlapping three dimensional surface representation may be trimmed along the central cut plane to expose additional surfaces along which measurements may be taken. For example, as illustrated in FIG. 16, the sag distance 258 between the bottom of the absorbent article of the first three dimensional surface representation and the bottom of the absorbent article of the second three dimensional surface representation may be obtained. This measurement may be useful to determine the sag of the absorbent article after the absorbent article has been prepared. It is to be appreciated that the sag distance 258 may also be measured between the crotch of the panelist and the absorbent article, or, in the case of three three dimensional surface representations the sag distance 258 may be measured between the crotch, a first absorbent article, and/or a second absorbent article. Further, in the overlapping three dimensional surface representation additional measurements may be taken such as the angle a between portions of the first three dimensional surface representation and the second three dimensional surface representation. This measurement may be useful in assessing how the appearance of the absorbent article changed based on the changes to the absorbent article, such as two different absorbent articles or an absorbent article that has undergone preparation as previously described.

It is to be appreciated that a cutting plane may be taken at any location that segments the first three dimensional surface representation, the second three dimensional surface representation, and/or the overlapping three dimensional surface representation. For instance, a cutting plane may be taken horizontally with respect to a feature present in the three dimensional surface representation, vertically with respect to a feature present in the three dimensional surface representation, or at an angle with respect to a feature present in the three dimensional surface representation. A feature is a visually identifiable element present in the three dimensional surface representation. An example of a feature may be a graphic disposed on the absorbent article, a side panel, an ear, a landing zone, a bulge of the absorbent article, sag, gapping, which may include the absorbent article separating from the waist or other portion of the panelist, an edge of the absorbent article, parts of the product protruding beyond the rest of the product or the surface of the panelist, parts of the absorbent article that have folded, rolled, twisted, distorted, etc., any type of marker that was placed on the absorbent article or panelist for the testing, or anatomical features like the navel, crotch, opening of the urethra, or the bending location of a knee. It is to be appreciated that the type of feature present in the three dimensional surface representation will depend on the consumer product that is to be imaged and the presence or absence of a panelist.

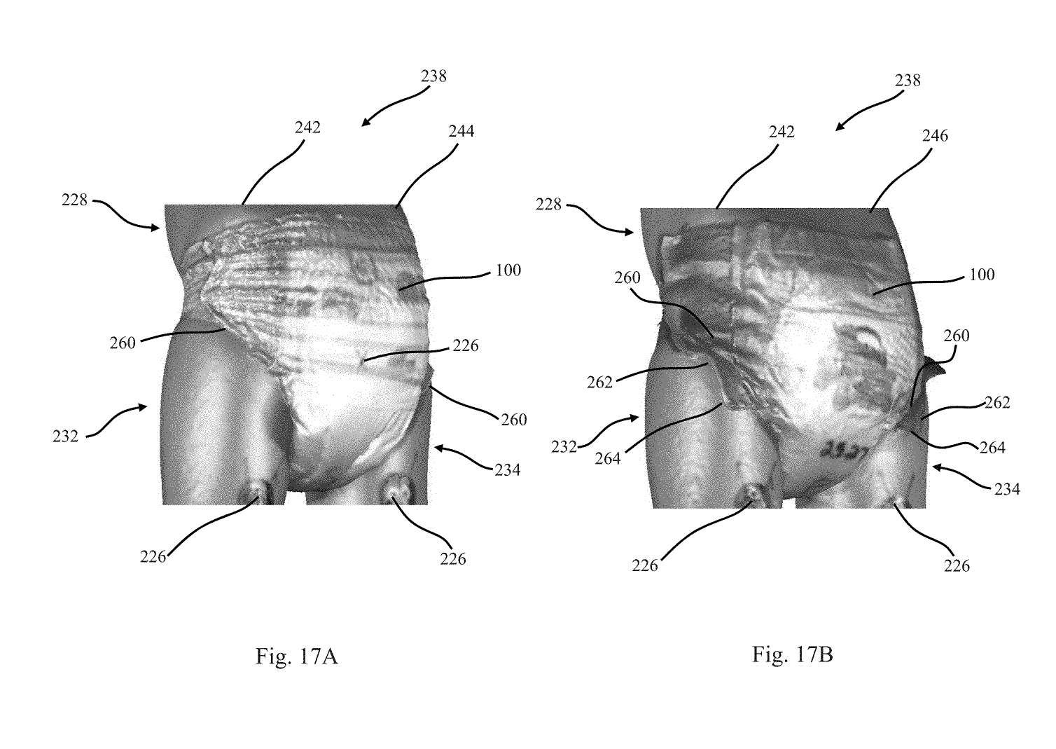

Measurements of individual features may be obtained using the overlapping three dimensional surface representation and/or each of the first three dimensional surface representation and the second three dimensional surface representation. For example, as illustrated in FIGS. 17A and 17B the difference in the coverage of a portion of the back of the leg opening of two different absorbent articles may be analyzed. To analyze the coverage of the portion of the back of the leg opening of the absorbent articles, a first trim line 260 may be inserted into the first three dimensional surface representation 244 that outlines the edge of the portion of the back of the leg opening of the first absorbent article or, stated differently, the boundary between the representation of the panelist's skin and the portion of the back of the leg opening of the first absorbent article. A second trim line 262 may be inserted on a portion of each of the leg openings, as illustrated in FIG. 17B. The second trim line 262 may be inserted into the second three dimensional surface representation 246 such that the second trim line 262 outlines the edge of the portion of the back of the leg opening of the second absorbent article or, stated differently, the boundary between the representation of the panelist's skin and the portion of the back of the leg opening of the second absorbent article. The length of the first trim line 260 and the length of the second trim line 262 may be determined and compared. Further, the first trim line 260 may intersect with the second trim line 262 to form a trim border 264, which may form a closed loop. The surface area within the trim border 264 may be determined such that the additional coverage area of the portion of the back of the leg opening of the absorbent article may be obtained. The perimeter or a portion of the perimeter of the trim border 264 may also be determined. The angle between the first trim line and the second trim line may also be determined.

The surface area of features present in the three dimensional surface representation may also be determined. For example, the difference in surface area of graphic coverage, landing zone coverage, and/or side panel coverage may be determined.

Features that are visible in one three dimensional surface representation may be located in the other three dimensional surface representation, but the feature may be hidden, partially hidden, or not present. It is to be appreciated that some features may not be identifiable until the first three dimensional surface representation is overlapped with the second three dimensional surface representation. For example, in a first three dimensional surface representation, a starting measurement point may be identified. The starting measurement point should have a corresponding, identifiable point in the second three dimensional surface representation. For example, the markers and sealed planes are likely present and identifiable in each of the first three dimensional surface representation and the second three dimensional surface representation. A first feature, such as a graphic, the lowest point of the crotch, etc., may be identified in the first three dimensional surface representation. The first feature that is identified in the first three dimensional surface representation may not be visible, or may be partially visible, or may not be present in the second three dimensional surface representation. A first measurement beginning at the starting measurement point and ending at the first feature or some predetermined distance is obtained. The first measurement may then be applied to the second three dimensional surface representation. More specifically, a corresponding starting measurement point is identified in the second three dimensional surface representation. The first measurement begins at the corresponding starting measurement point and identifies an end point on the second three dimensional surface representation. It is to be appreciated that the direction in which the first measurement extends from the starting measurement point to the feature may be repeated in the second three dimensional surface representation to accurately identify the intended location in the second three dimensional surface representation.

Various measurements may be obtained using the end point. For example, a measurement plane may be placed at the end point and the portion of the second three dimensional surface representation to either one of the sides of the plane may be trimmed and thus, removed, such that a volume measurement may be obtained. In some embodiments, an area surrounding the end point may be selected such that a surface area, such as in the case of graphic coverage, may be obtained. Similarly, the perimeter or a portion of the perimeter of the area surrounding the end point may be selected. Further, the end point may become the next point off of which measurements may be taken. For example, measurements may be taken from the end point to the bottom of the absorbent article or to the top of the absorbent article. Further still, the end point may be disposed on a feature, which may be a second feature, present in the second three dimensional surface representation. The characteristics, such as surface area and/or at least a portion of the perimeter, of the first feature and the second feature may be compared.

It is to be appreciated that the pixel information of a feature may be obtained from the three dimensional surface representation. The pixel information may aid in evaluating the change in appearance of the absorbent article. For example, the appearance, such as brightness, surface area, contrast, and visibility, of a graphic may be determined based on the pixel information.

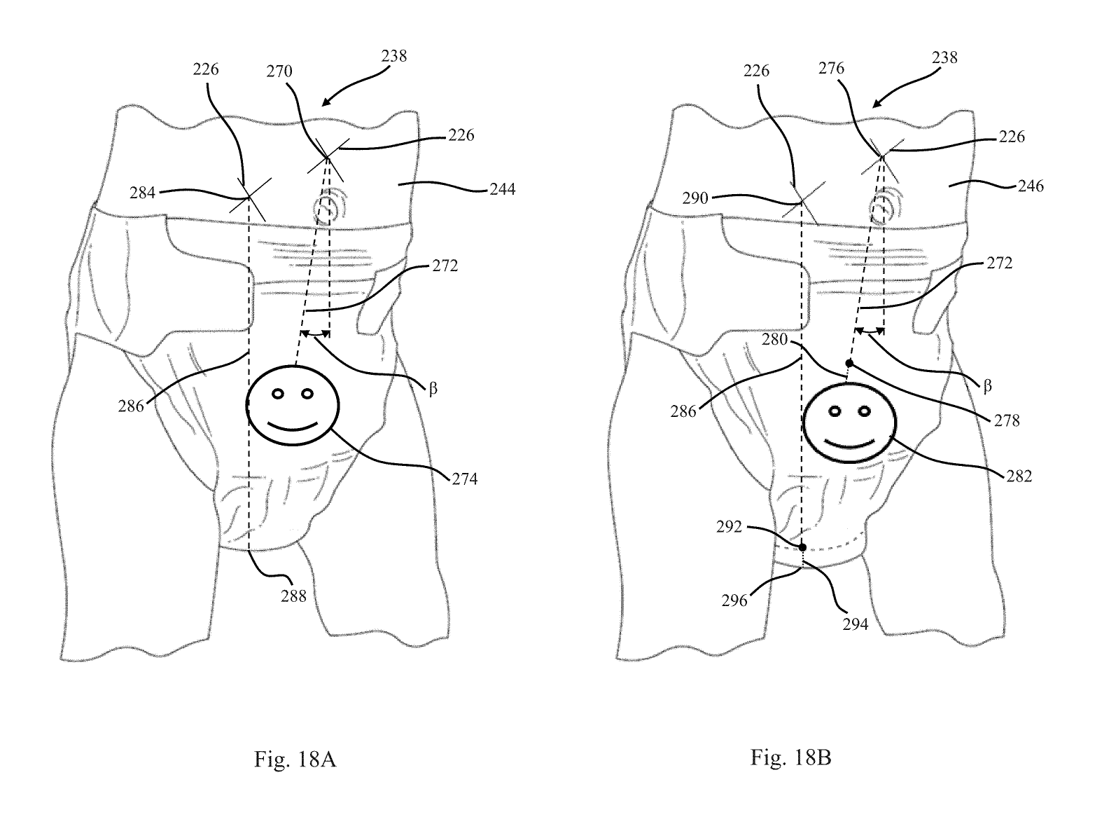

Referring to FIGS. 18A and 18B, for example, the location of a feature, such as a graphic or the bottom of the crotch portion may be compared. A starting measurement point 270 may be identified in a first three dimensional surface representation 244. A first measurement 272 beginning at the starting measurement point 270 and ending at a feature 274 or some predetermined distance is obtained. The first measurement 272 may be taken at an angle β, as illustrated in FIG. 18A. It is to be appreciated that the first measurement 272 may also include the addition of one or more measurements taken horizontally and/or vertically. The first measurement 272 may then be applied to the second three dimensional surface representation 246. More specifically, a corresponding starting measurement point 276 is identified in the second three dimensional surface representation 246. The first measurement 272 begins at the corresponding starting measurement point 276 and identifies an end point 278 on the second three dimensional surface representation 246. It is to be appreciated that the direction in which the first measurement extends from the starting measurement point to the feature may be repeated in the second three dimensional surface representation. As illustrated in FIG. 18B, the first measurement 272 is again taken at the angle β. The end point 278 may provide a second starting measurement point such that the two three dimensional surface representations may be compared. As illustrated in FIG. 18B, a second measurement 280 may extend from the end point 278 to the second feature 282 present in the second three dimensional surface representation 246 to obtain the difference in location of the features of the first and second three dimensional surface representations.

Similarly, the difference in the location of the crotch portion of the absorbent article may be obtained from the first and second three dimensional surface representations. A second starting measurement point 284 may be identified in a first three dimensional surface representation 244. A third measurement 286 beginning at the second starting measurement point 284 and ending at a third feature 288, such as the bottom of the crotch portion of the absorbent article, or some predetermined distance is obtained. The first measurement 272 may be taken in a substantially vertical direction, as illustrated in FIG. 18A. The third measurement 286 may then be applied to the second three dimensional surface representation 246. More specifically, a second corresponding starting measurement point 290 is identified in the second three dimensional surface representation 246. The third measurement 286 begins at the second corresponding starting measurement point 290 and identifies a second end point 292 on the second three dimensional surface representation 246. It is to be appreciated that the direction in which the first measurement extends from the starting measurement point to the feature may be repeated in the second three dimensional surface representation. The second end point 292 may provide a fourth starting measurement point such that the two three dimensional surface representations may be compared. As illustrated in FIG. 18B, a fourth measurement 294 may extend from the end point 292 to the fourth feature 296 present in the second three dimensional surface representation 246 to obtain the difference in location of the features of the first and second three dimensional surface representations.

In some other embodiments, the overlapping three dimensional surface representations may be individually brought to the most outer surface of the overlapping three dimension surface representation. For example, if the overlapping three dimensional surface representation includes a first three dimensional surface representation overlapped with a second three dimensional surface representation, the first three dimensional surface representation may be selected such that features on the first three dimensional surface representation are visible and may be identified for use in measurements. Similarly, the second three dimensional surface representation may be selected such that features on the second three dimensional surface representation are visible and may be identified for use in measurements and the first three dimensional surface representation may be hidden or unselected. In some embodiments, the first three dimensional surface representation may be selected such that the features present in the first three dimensional surface representation are most visible. A starting measurement point may be identified in the first three dimensional surface representation and an indicator, such as a mark, may be made at the location of the starting point. The second three dimensional surface representation may then be selected such that the features present in the second three dimensional surface representation come to the forefront of the overlapping three dimensional surface representations. The starting measurement point that was previously indicated in the first three dimensional surface representation may remain visible on the second three dimensional surface representation as it is selected and brought forward. The starting measurement point although initially identified using the first three dimensional surface representation may be used for measurements in the second three dimensional surface representation.

More specifically, for example, the first three dimensional surface representation may include a naked panelist and the second three dimensional surface representation may include the panelist wearing an absorbent article. The first three dimensional surface representation may be overlapped with the second three dimensional surface representation. While the first and second three dimensional surface representations are overlapped, the first three dimensional surface representation may be selected such that it is brought to the forefront and features of the first three dimensional surface representation are visible. The crotch of the panelist may be identified in the first three dimensional surface representation. A mark or some other visible indication of the bottom of the crotch may be added to the overlapping three dimensional surface representation. The second three dimensional surface representation may then be selected or brought to the forefront such that the features of the second three dimensional surface representation become visible. The visible indication that was previously added remains visible even when the second three dimensional surface representation is selected or brought to the forefront and this visible indication of the crotch may become the starting measurement point or an ending measurement point. A measurement starting or ending at the visible indication of the crotch may be made in the second three dimensional surface representation. It is to be appreciated that greater than two three dimensional surface representations may be used to form an overlapping three dimensional surface representation and any feature may be used to make a visible indication that may be used as a starting or ending measurement point in one or more other three dimensional surface representations. It is also to be appreciated that the panelist may be wearing an absorbent article in multiple three dimensional surface representations that have been overlapped.

It is also to be appreciated that the aforementioned measurements may be taken on a three dimensional surface representation that has not undergone trimming and sealing. Trimming and sealing are necessary when the measurement to be obtained requires a watertight surface representation, such as for obtaining volume of the surface representation.

The aforementioned methods are useful for evaluating product fit. For example, where the product is a diaper, fit can be evaluated before, during, and after single or multiple liquid depositions; as the child is in different positions; as the diaper is secured by a caretaker in different ways (tightness, position of tape on landing zone, location on hips, etc.), and subsets and combinations of the above.

It should now be understood that various three dimensional surface representations may be compared. When testing and designing products, the three dimensional surface representations of the products may be used to analyze performance characteristics such as fit and/or appearance.

Where the product is an absorbent article such as a diaper, these methods also enable evaluation of fit as the product is worn, before, during, and/or after one or more depositions of a fluid, a solid or a semi-solid material into the absorbent article. The methods also enable evaluation of how appearance is affected by wear of the article, user movement, deposition into the article of fluids, solids, and semi-solids, other conditions, and combinations of these. The information obtained may be used in any of a variety of ways. Where the product is an absorbent article, such as a diaper, the information enhances evaluation and development of the article's fit and appearance.

The information also may enhance product design. For example, the goal of an article, as for a young baby, may be to keep the child dry, comfortable, and for the absorbent article to fit well. In contrast, where the article is intended for use with an older child who may be in the potty training stage, the purpose of the article may be to contain exudates and prevent leaks, while simultaneously maintaining some level of contact between the child and the exudates to prevent the child from being overly comfortable after soiling the article, and to provide training signals to the child such as through fading and appearing graphics and/or sensorial signals such as a feeling of temperature change. The techniques of the present disclosure provide information relating to how fluids, semi-solids, and solids effect the diaper as it is worn, which may be useful to the product's designers.

Although the present disclosure makes reference to particular examples, persons skilled in the art will recognize that changes may be made in form and detail without departing from the spirit and scope of the disclosure. All directional references (e.g., rear, front, left, right, top, bottom) are only used for identification purposes to aid the reader's understanding of the examples of the present disclosure. It is intended that all matter contained in the above description or shown in the accompanying drawings shall be interpreted as illustrative only and not as limiting. Changes in detail or structure may be made without departing from the spirit of the disclosure as defined in the appended claims.

A “computer,” “computer system,” or “processor” may be, for example and without limitation, a processor, microcomputer, minicomputer, server, mainframe, laptop, personal data assistant (PDA), wireless e-mail device, cellular phone, pager, processor, fax machine, scanner, or any other programmable device configured to transmit and/or receive data over a network and capable of executing machine readable instructions. It is noted that the examples described herein may comprise distributed computing devices. Specifically, multiple autonomous processors may be communicably coupled and configured to cooperate in a manner analogous to the single processor. Computer systems and computer-based devices disclosed herein may include memory for storing certain software applications used in obtaining, processing, and communicating information. It is be appreciated that such memory may be internal or external with respect to operation of the disclosed embodiments. The memory may also include any means for storing software, including a hard disk, an optical disk, floppy disk, ROM (read only memory), RAM (random access memory), PROM (programmable ROM), EEPROM (electrically erasable PROM) and/or other computer-readable memory media.

In general, it will be apparent to one of ordinary skill in the art that various embodiments described herein, or components or parts thereof, may be implemented in many different embodiments of software, firmware, and/or hardware, or modules thereof. The software code or specialized control hardware used to implement some of the present embodiments is not limiting of the present disclosure. For example, the embodiments described hereinabove may be implemented in computer software using any suitable computer programming language such as .NET, SQL, MySQL, or HTML using, for example, conventional or object-oriented techniques. Programming languages for computer software and other computer-implemented instructions may be translated into machine language by a compiler or an assembler before execution and/or may be translated directly at run time by an interpreter. Examples of assembly languages include ARM, MIPS, and x86; examples of high level languages include Ada, BASIC, C, C++, C#, COBOL, Fortran, Java, Lisp, Pascal, Object Pascal; and examples of scripting languages include Bourne script, JavaScript, Python, Ruby, PHP, and Perl. Such software may be stored on any type of suitable computer-readable medium or media such as, for example, a magnetic or optical storage medium. Thus, the operation and behavior of the embodiments are described without specific reference to the actual software code or specialized hardware components. The absence of such specific references is feasible because it is clearly understood that artisans of ordinary skill would be able to use software and control hardware to implement the embodiments of the present disclosure based on the description herein with only a reasonable effort and without undue experimentation. Various embodiments of the systems and methods described herein may employ one or more electronic computer networks to promote communication among different components, transfer data, or to share resources and information. Such computer networks can be classified according to the hardware and software technology that is used to interconnect the devices in the network, such as optical fiber, Ethernet, wireless LAN, HomePNA, or power line communication. The computer networks may also be embodied as one or more of the following types of networks: local area network (LAN); metropolitan area network (MAN); wide area network (WAN); virtual private network (VPN); storage area network (SAN); or global area network (GAN), among other network varieties.

In various embodiments, computers and computer systems described herein may have the following main components: arithmetic and logic unit (ALU), control unit, memory, and input and output devices (I/O devices). These components can be interconnected by busses, often comprising groups of wires or cables. The control unit, ALU, registers, and basic I/O (and often other hardware closely linked with these sections) can be collectively considered a central processing unit (CPU) for the computer system. The CPU may be constructed on a single integrated circuit or microprocessor.

This application claims the benefit of U.S. Provisional Application No. 62/340,572 filed on May 24, 2016, the entirety of which is incorporated by reference herein.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

All documents cited in the Detailed Description are, in relevant part, incorporated herein by reference, however the citation of any document is not construed as an admission that it is prior art with respect to the present disclosure. To the extent that any meaning or definition of a term in this written document conflicts with any meaning or definition of the term in a document incorporated by reference, the meaning or definition assigned to the term in this written document shall govern.

While particular examples have been illustrated and described herein, it should be understood that various other changes and modifications may be made without departing from the spirit and scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

What is claimed is:

1. A method of analyzing fit and appearance of an absorbent article, the method comprising:
marking a panelist using a marking protocol, wherein the marking protocol comprises placement of three or more markers on an external surface of the panelist;
positioning the panelist in an orientation;
imaging the external surface of the panelist with an image capture device, wherein the image capture device uses visible light or an infrared light;
generating a first three dimensional surface representation of the surface of the panelist using a computer system;
preparing an absorbent article;
imaging the external surface of the panelist wearing the absorbent article with the image capture device;
generating a second three dimensional surface representation of the surface of the panelist wearing the absorbent article using the computer system;
identifying at least three of the three or more markers present in the first three dimensional surface representation of the panelist;
selecting the at least three of the three or more markers present in the first three dimensional surface representation using the computer system;
identifying at least three of the three or more markers present in the second three dimensional surface representation of the panelist wearing the absorbent article;
selecting the at least three of the three or more markers present in the second three dimensional surface representation using the computer system;
aligning, using the computer system, at least three of the three or more markers of the first three dimensional surface representation with at least three of the three or more markers of the second three dimensional surface representation to form an overlapping three dimensional surface representation; and
comparing the first three dimensional surface representation and the second three dimensional surface representation.

2. The method of claim 1, wherein the marking protocol comprises:
identifying three or more markers that contrast compared to at least one of the external surface of the panelist and the external surface of the absorbent article;
placing a first marker set comprising at least three markers on at least one of the external surface of the panelist and the external surface of the absorbent article to define a first plane in a first location.

3. The method of claim 2, wherein the marking protocol comprises:
placing a second marker set comprising at least three markers on at least one of the external surface of the panelist and the external surface of the absorbent article to define a second plane in a second location;
placing a third marker set comprising at least three markers on at least one of the external surface of the panelist and the external surface of the absorbent article to define a third plane in a third location; and
placing a fourth mark on a centerline of the external surface of the panelist.

4. The method of claim 2, wherein the first location is a torso region of the panelist.

5. The method of claim 3, wherein the second location is a first thigh region of the panelist, and wherein the third location is a second thigh region of the panelist.

6. The method of claim 1, wherein each of the three or more markers is a three dimensional object or a two dimensional mark.

7. The method of claim 1, wherein the three or more markers are visible in at least one of a texture map, the first three dimensional surface representation, and the second three dimensional surface representation.

8. The method of claim 1, comprising marking the absorbent article using one or more markers.

9. The method of claim 1, comprising distorting at least one of the first three dimensional surface representation and the second three dimensional surface representation such that the first three dimensional surface representation is positioned in substantially the same orientation as the second three dimensional surface representation.

10. The method of claim 1, comprising repositioning the panelist in the orientation.

11. The method of claim 1, wherein preparing the absorbent article comprises placing the absorbent article on the panelist.

12. The method of claim 1, comprising repairing the first three dimensional surface representation and repairing the second three dimensional surface representation.

13. The method of claim 1, comprising:
repairing the first three dimensional surface representation;
repairing the second three dimensional surface representation;
identifying at least three of the three or more markers on the first three dimensional surface representation to form a first cutting plane;
identifying at least three of the three or more markers on the first three dimensional surface representation to form a second cutting plane;
trimming the first three dimensional surface representation along the first cutting plane and the second cutting plane;
identifying at least three of the three or more markers on the second three dimensional surface representation to form a third cutting plane;
identifying at least three of the three or more markers on the second three dimensional surface representation to form a fourth cutting plane;
trimming the second three dimensional surface representation along the third cutting plane and the fourth cutting plane;
sealing the first three dimensional surface representation along the first cutting plane and the second cutting plane such that the first three dimensional surface representation is watertight; and
sealing the second three dimensional surface representation along the third cutting plane and the fourth cutting plane such that the second three dimensional surface representation is watertight.

14. The method of claim 1, comprising:
repairing the first three dimensional surface representation;
repairing the second three dimensional surface representation;
identifying at least three of the three or more markers on the overlapping three dimensional surface representation to form a first cutting plane;
trimming the overlapping three dimensional surface representation along the first cutting plane;
identifying at least three of the three or more markers on the overlapping three dimensional surface representation to form a second cutting plane;
trimming the overlapping three dimensional surface representation along the second cutting plane;
sealing the overlapping three dimensional surface representation along the first cutting plane; and
sealing the overlapping three dimensional surface representation along the second cutting plane such that the overlapping three dimensional surface representation is watertight.

15. The method of claim 13, wherein comparing the first three dimensional surface representation and the second three dimensional surface representation comprises obtaining the difference in volume between the first three dimensional surface representation and the second three dimensional surface representation.

16. The method of claim 13, wherein comparing the first three dimensional surface representation and the second three dimensional surface representation comprising:
dividing the first three dimensional surface representation to form a first portion and a second portion;
dividing the second three dimensional surface representation to form a first portion and a second portion;
comparing the first portion of the first three dimensional surface representation and first portion of the second three dimensional surface representation; and
comparing the second portion of the first three dimensional surface representation and the second portion of the second three dimensional surface representation.

17. The method of claim 12, wherein comparing the first three dimensional surface representation and the second three dimensional surface representation comprising:
identifying a first feature in the first three dimensional surface representation;
identifying a second feature in the second three dimensional surface representation;
tracing a portion of the first feature to form a first trim line;
tracing a portion of the second feature to form a second trim line;

18. The method of claim 17, comprising:
connecting the first trim line and the second trim line to form a trim border;
obtaining the surface area defined by the trim border.

19. The method of 17, comprising:
connecting the first trim line and the second trim line to form a trim border;
obtaining the length of the trim border.

20. The method of 17, wherein the first trim line and the second trim line intersect forming an angle.

21. The method of claim 13, comprising:
filling the watertight first three dimensional surface representation; and
filling the watertight second three dimensional surface representation.

22. The method of claim 1, wherein comparing the first three dimensional surface representation and the second three dimensional surface representation comprising:
identifying a starting measurement point in the first three dimensional surface representation, wherein the starting measurement point is identifiable in the second three dimensional surface representation;
identifying a first feature in the first three dimensional surface representation, wherein the first feature is not visible in the second three dimensional surface representation;
obtaining a first measurement between the starting measurement point and the first feature;
applying the first measurement to the second three dimensional surface representation, wherein the first measurement begins at a corresponding starting measurement point in the second three dimensional surface representation; and
identifying an end point of the first measurement on the second three dimensional surface representation.

23. The method of claim 22, comprising:
forming a measurement plane at the end point of the first measurement on the second three dimensional surface representation;
trimming the second three dimensional surface representation such that the portion of the second three dimensional surface representation above or below the measurement plane is removed to form a trimmed second three dimensional surface representation; and
obtaining a volume, surface area, or length of at least a portion of the perimeter of the trimmed second three dimensional surface representation.

24. The method of claim 22, comprising selecting a second feature on the second three dimensional surface adjacent to or overlapping the end point of the first measurement.

25. The method of claim 24, comprising obtaining the surface area of the second feature.

26. The method of claim 24, comprising obtaining the length of at least a portion of the perimeter of the second feature.

27. The method of claim 22, comprising obtaining the pixels of at least a portion of the second feature.

28. The method of claim 1 comprising:
imaging the external surface of the panelist wearing a second absorbent article with the image capture device; and
generating a third three dimensional surface representation of the surface of the panelist wearing the second absorbent article using the computer system.

29. The method of claim 1 comprising:
preparing the absorbent article for a second time;
imaging the external surface of the panelist wearing the absorbent article with the image capture device; and
generating a third three dimensional surface representation of the surface of the panelist wearing the absorbent article using the computer system.

30. A method of analyzing fit of an absorbent article disposed on a panelist, the method comprising:
preparing a first absorbent article;
marking a panelist wearing the first absorbent article using a marking protocol, wherein the marking protocol includes placement of three or more markers on an external surface of at least one of the panelist and the first absorbent article;
positioning the panelist in an orientation;
imaging, using an image capture device and a computer system, the external surface of the panelist wearing the first absorbent article to generate one or more first images, wherein the image capture device uses a visible light and/or an infrared light;
using a computer modeling system to generate a first three dimensional surface representation of the surface of the panelist from the one or more first images;
preparing a second absorbent article;
imaging, using the image capture device and the computer system, the external surface of the panelist wearing the second absorbent article to generate one or more second images;
using the computer modeling system to generate a second three dimensional surface representation of the surface of the panelist wearing the second absorbent article from the one or more second images;
identifying the three or more markers present in the first three dimensional surface representation of the panelist wearing the first absorbent article;
identifying the three or more markers present in the second three dimensional surface representation of the panelist wearing the second absorbent article;
aligning, using the computer modeling system, the three or more markers of the first three dimensional surface representation with the three or more markers of the second three dimensional surface representation to form an overlapping three dimensional surface representation; and
comparing the first three dimensional surface representation and the second three dimensional surface representation.

31. The method of claim 30, wherein the first absorbent article is a different brand than the second absorbent article.

32. The method of claim 30 comprising:
imaging, using the image capture device and the computer system, the external surface of the panelist wearing the second absorbent article to generate one or more third images;
using the computer modeling system to generate a third three dimensional surface representation of the surface of the panelist wearing the second absorbent article from the one or more third images;
identifying three or more markers present in the third three dimensional surface representation of the panelist wearing the second absorbent article;
aligning, using the computer modeling system, the three or more markers of the first three dimensional surface representation with the three or more markers of the second three dimensional surface representation and the three or more markers of the third three dimensional surface representation to form an overlapping three dimensional surface representation; and
comparing at least two of the first three dimensional surface representation, the second three dimensional surface representation, and the third three dimensional surface representation.

33. A method of analyzing fit of an absorbent article disposed on a panelist, the method comprising:
marking a panelist wearing a first absorbent article using a marking protocol, wherein the marking protocol includes placement of three or more markers on an external surface of at least one of the panelist and the first absorbent article;
positioning the panelist in an orientation;
imaging the external surface of the panelist wearing the first absorbent article using an image capture device to generate one or more first images, wherein the image capture device comprises one or more digital cameras, and wherein the image capture device uses visible light and/or infrared light;
generating a first three dimensional surface representation of the surface of the panelist using a computer system and the one or more first images;
preparing the first absorbent article;
imaging the external surface of the panelist wearing the first absorbent article using the image capture device to generate one or more second images;
generating a second three dimensional surface representation of the surface of the panelist wearing the first absorbent article using the computer system and the one or more second images;
identifying the three or more markers present in the first three dimensional surface representation of the panelist;
identifying the three or more markers present in the second three dimensional surface representation of the panelist wearing the first absorbent article;
aligning, using the computer system, the three or more markers of the first three dimensional surface representation with the three or more markers of the second three dimensional surface representation to form an overlapping three dimensional surface representation; and
comparing the first three dimensional surface representation and the second three dimensional surface representation.

34. The method of claim 33 comprising:
preparing a second absorbent article;
imaging the external surface of the panelist wearing the second absorbent article using the image capture device to generate one or more third images;
generating a third three dimensional surface representation of the surface of the panelist wearing the second absorbent article using the computer system and the one or more third images;
identifying the three or more markers present in the third three dimensional surface representation of the panelist wearing the second absorbent article;
aligning, using the computer system, the three or more markers of the first three dimensional surface representation with the three or more markers of the second three dimensional surface representation and with the three or more markers of the third three dimensional surface representation to form an overlapping three dimensional surface representation; and
comparing the first three dimensional surface representation, the second three dimensional surface representation, and the third three dimensional surface representation.
